(19) United States
(12) Patent Application Publication
(10) Pub. No.: US 2006/0217370 A1
(43) Pub. Date: Sep. 28, 2006

(54) Compounds useful for the treatment and prevention of pain and screening methods therefor

(76) Inventors: Ethan Burstein, San Diego, CA (US); Leila Malik, Copenhagen (DK); Nicholas Kelly, Bagsvaerd (DK)

Correspondence Address: SQUIRE, SANDERS & DEMPSEY L.L.P., 1 MARITIME PLAZA, SUITE 300, SAN FRANCISCO, CA, 94111, US

(21) Appl. No.: 11/359,209

(22) Filed: Feb. 21, 2006

Related U.S. Application Data
(60) Provisional application No. 60/654,539, filed on Feb. 18, 2005.

(51) Int. Cl. A61K 31/551; C07D 487/22

(52) U.S. Cl. 514/219; 540/494; 540/555

(57) ABSTRACT

The present invention relates to the composition of compounds having the generic structure: 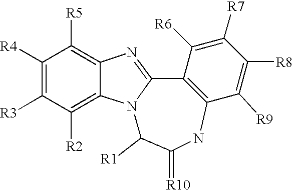 and to a method of treatment or prevention of pain using the above compounds.

### RELATED APPLICATIONS

This application claims the benefit of Provisional Patent Application Ser. No. 60/654,539, filed on 18 Feb. 2005. The '539 application is incorporated by reference, including drawings, as if fully set forth herein.

### FIELD

The present invention relates to the fields of organic chemistry, pharmaceutical chemistry, biochemistry, molecular biology and medicine. More particularly, it relates to compounds that are useful for the treatment and prevention of pain and to methods of screening for them.

### BACKGROUND

Opiates are currently the most extensively used compounds for the clinical treatment of pain (Reisine and Pasternak, 1996). The opiates, however, have a number of side effects that limit their therapeutic use. They can cause respiratory depression and nausea and, of course, they are addictive. The side effects are primarily due to their activity at central sites in the brain. It is believed that compounds that interact with targets in peripheral pain pathways rather than the central nervous system may be capable of reducing pain with minimal side effects. The dorsal root ganglia constitute such a target.

The DRG contain afferent neurons known as nociceptive neurons that respond to acute and chronic pain stimuli in the peripheral organs and transmit signals to the central nervous system causing the sensation of pain. Drugs that target nociceptive sensory neurons in the DRG could potentially block pain transmission with the desired minimal side effects including, in particular, physical dependency.

Recent studies have identified a family of G-protein coupled receptors (GPCRs) known as sensory neuron specific GPCRs (SNSRs) that are primarily expressed in DRG nociceptive neurons. These receptors are structurally similar to the Mas oncogene, which is also a GPCR and are also referred to as Mas related genes (Mrgs) (Dong et al., 2001; Zylka et al., 2003). In addition, certain endogenous peptides have been identified that interact with SNSRs to produce analgesia in rodents (Hong et al., 2004; Lembo et al., 2002; Han et al., 2002, Robas et al., 2003; Grazzini et al., 2004). For example, MrgA1 and MrgC11 are activated by RF-amide related peptides (Han et al., 2002) which blocks pain transmission when administered intrathecally (Panula et al. 1996, 1999). Similarly, MrgX1, which in humans is only expressed in DRG, is potently stimulated in vitro by the opioid peptide bovine adrenal medulla peptide 22 (BAM22). BAM22 is believed to induce analgesia through mechanisms independent of opiate receptor stimulation (Hong et al. 2004). These studies and others suggest that the Mrgs may be useful targets for the development of novel analgesics.

A problem with working with MrgX receptors, however, is that no homologues of them exist in rodents (see Dong et al, 2001) rendering them ineffective as animal models for testing.

What is needed are compounds that interact with afferent nociceptive MrgX neurons to treat or prevent pain. A method to screen for such compounds and an animal model with which to validate the therapeutic potential of candidate compounds is also needed. The present invention provides each of these.

### SUMMARY

Following are some of the aspects of this invention. The aspects presented below are not intended, nor are they to be construed, as limiting the scope of this invention in any manner. Other aspects will become apparent based on the entire disclosure herein.

Thus, an aspect of this invention is a compound that selectively modulates the activity of MrgX1 and MrgX2 receptors relative to other receptors that mediate analgesia such as the opioid receptors. In another aspect, this invention relates to methods of alleviating acute, chronic and neuropathic pain caused by, without limitation, trauma, various diseases and disorders and the side effects of other drug therapies, by administering to a patient in need thereof a therapeutically effective amount of a compound of this invention. Methods of modulating the activities of MrgX1 and MrgX2 receptors by administering an effective amount of a compound of this invention are also an aspect of this invention. In addition, the present invention relates to isolated polynucleotides encoding novel simian homologues of MrgX1 and MrgX2 receptors and functional equivalents and fragments thereof. Furthermore, this invention relates to the use of these polynucleotides in the identification of compounds with therapeutic utility at the human receptors. In a further aspect of this invention, a vector containing novel GPCR polynucleotide sequences is provided. In another aspect of this invention, the vector is an expression vector. The expression vector may be present in a host cell, such as bacterial, yeast, insect, plant, or animal cell.

An aspect of this invention comprises a method of screening for a compound effective for the treatment or prevention of pain comprising contacting a test compound with a receptor selected from the group consisting of a human MrgX1 receptor, a simian MrgX1 receptor, a human MrgX2 receptor and a simian MrgX2 receptor and detecting changes in the activity of the receptor.

An aspect of this invention comprises a method of screening for a compound that modulates the activity of a human MrgX1 receptor, a simian MrgX1 receptor, a human MrgX2 receptor or a simian MrgX2 receptor comprising providing a cell, a plurality of cells or an extract that express(es) the receptor, contacting the cell(s) or extract with a test compound and detecting changes in the activity of the receptor. Contacting a cell or plurality of cells may comprise incubating the cell(s) with the test compound. The cell(s) may be engineered to over-express the receptor. The assay may further comprise the addition of an known agonist to the test milieu to assist in differentiating an antagonist from an inverse agonist. In general, if the activity of the receptor is increased, the compound is an agonist, if the basal activity of the receptor, as measured before any compound is added, is decreased, the compound is likely an inverse agonist while if the receptor is inactivated, the compound is an antagonist.

An aspect of this invention comprises a method of treating or preventing pain, comprising administering to a patient in need thereof an effective amount of at least one compound that modulates the activity of a human MrgX1 receptor, a simian MrgX1 receptor, a human MrgX2 receptor or a simian MrgX2 receptor.

An aspect of this invention is a compound having the chemical formula: 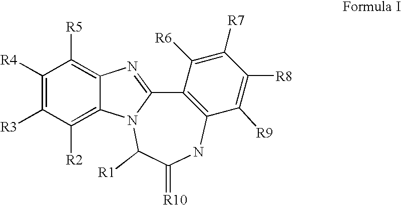
wherein:
R₁ is selected from the group consisting of hydrogen, unsubstituted or substituted alkyl, unsubstituted or substituted alkenyl, unsubstituted or substituted phenyl, unsubstituted or substituted naphthyl, unsubstituted or substituted phenylalkyl, unsubstituted or substituted heteroaryl, unsubstituted or substituted heteroalicyclyl, cyano, unsubstituted or substituted acyl, unsubstituted or substituted alkoxycarbonyl, unsubstituted or substituted alkylaminocarbonyl, unsubstituted or substituted phenylaminocarbonyl, unsubstituted or substituted alkylthio, unsubstituted or substituted alkylsulfonyl, unsubstituted or substituted alkylsulfinyl, unsubstituted or substituted phenylthio, unsubstituted or substituted phenylsulfinyl, unsubstituted or substituted phenylsulfonyl and unsubstituted and substituted or unsubstited cycloalkyl;
R₂, R₃, R₄, R₅, R₆, R₇, R₈, R₉ are independently selected from the group consisting of hydrogen, hydroxy, protected hydroxy, unsubstituted or substituted alkyl, unsubstituted or substituted alkenyl, unsubstituted or substituted alkynyl, unsubstituted or substituted cycloalkyl, unsubstituted or substituted cycloalkenyl, unsubstituted or substituted aryl, unsubstituted or substituted heteroaryl, unsubstituted or substituted heteroalicyclyl, unsubstituted or substituted heteroarylalkyl, unsubstituted or substituted heteroalicyclylalkyl, halogenated ether, nitro, amino, halogen, perhaloalkyl, —NX₁ₐX_{1b}, —CN, —C(=Z)X₁ₐ, —C(=Z)OX₁ₐ, —C(=Z)NX₁ₐX_{1b}, —N(X₁ₐ)—C(=Z)X_{1b}, —N(X₁ₐ)—C(=Z)NX_{1b}X_{1c}, —OC(=Z)X₁ₐ, —S(═O)X₁ₐ, —S(═O)₂X₁ₐ, and —SX₁ₐ, and,
R₁₀ is selected from the group consisting or oxygen, —NH or —NX_{1d} wherein Z is oxygen or sulfur and X₁ₐ, X_{1b}, X_{1c} and X_{1d} are independently selected from the group consisting of hydrogen, substituted or unsubstituted aryl, substituted or unsubstituted heteroaryl, unsubstituted or substituted heteroalicyclyl, unsubstituted or substituted alkyl, unsubstituted or substituted alkenyl, unsubstituted or substituted alkynyl, unsubstituted or substituted cycloalkyl and unsubstituted or substituted cycloalkenyl.

An aspect of this invention relates to a compound of this invention wherein:
R₁ is selected from the group consisting of hydrogen, unsubstituted or substituted C₁ to C₆ alkyl, unsubstituted or substituted phenyl, unsubstituted or substituted phenylalkyl, unsubstituted or substituted heteraryl, unsubstituted or substituted heteroalicyclyl, unsubstituted or substituted alkoxycarbonyl, unsubstituted or substituted C₁ to C₁₂ alkylaminocarbonyl, unsubstituted or substituted phenylaminocarbonyl, unsubstituted or substituted C₁ to C₁₀ alkylthio, unsubstituted or substituted phenylthio, and unsubstituted or substituted C₅ to C₇ cycloalkenyl.

An aspect of this invention relates to a compound of this invention wherein:
R₂, R₃, R₄ and R₅ are independently selected from the group consisting of hydrogen, halogen, hydroxy, protected hydroxy, cyano, unsubstituted or substituted C₁ to C₁₂ alkyl, unsubstituted or substituted-C₁ to C₁₂ alkoxy, carboxy, protected carboxy, unsubstituted or substituted C₁ to C₁₀ alkylthio, —C(=Z)NX₁ₐX_{1b} and —C(=Z)X₁ₐ, wherein:
Z is oxygen or sulfur and X₁ₐ and X_{1b} together with the nitrogen atom to which they are covalently bonded form an unsubstituted or substituted heteroaryl or an unsubstituted or substituted heteroalicyclyl, or X₁ₐ and X_{1b} are independently selected from the group consisting of hydrogen, unsubstituted or substituted C₁ to C₁₂ alkyl, unsubstituted or substituted phenyl, unsubstituted or substituted phenylalkyl, unsubstituted or substituted heteroaryl(C₁ to C₁₂)alkyl, unsubstituted or substituted heteroaryl, unsubstituted or substituted heteroalicyclyl(C₁ to C₁₂)alkyl and unsubstituted or substituted heteroalicyclyl;
R₆, R₇, R₈ and R₉ are independently selected from the group consisting of hydrogen, halogen, unsubstituted or substituted heteroaryl, unsubstituted or substituted heteroalicycly, —NX₁ₐX_{1b} and —SX₁ₐ, wherein X₁ₐ and X_{1b} are independently selected from the group consisting of hydrogen, unsubstituted or substituted C₁ to C₁₂ alkyl, unsubstituted or substituted C₂ to C₁₂ alkenyl, unsubstituted or substituted phenylalkyl, unsubstituted or substituted heteroaryl, unsubstituted or substituted heteroalicyclyl; and
R₁₀ s selected from the group consisting of oxygen, —NH or —NX_{1d} where X_{1d} is selected from the group consisting of hydrogen, substituted or unsubstituted aryl, substituted or unsubstituted heteroaryl, unsubstituted or substituted C₁ to C₁₀ alkyl, unsubstituted or substituted C₂ to C₁₀ alkenyl, unsubstituted or substituted C₂ to C₁₀ alkynyl, unsubstituted or substituted C₃ to C₈ cycloalkyl and unsubstituted or substituted C₅ to C₈ cycloalkenyl.

An aspect of this invention relates a compound of this invention wherein:
R₂, R₃, and R₅ are each hydrogen; and,
R₄ is —C(=Z)NX₁ₐX_{1b} or —C(=Z)X₁ₐ, wherein:
Z is oxygen or sulfur and X₁ₐ and X_{1b} taken together with the nitrogen to which they are covalently bonded form a heterocycle or substituted heterocycle or X₁ₐ and X_{1b} are independently selected from the group consisting of hydrogen, unsubstituted or substituted C₁ to C₁₂ alkyl, unsubstituted or substituted phenyl, unsubstituted or substituted heteroaryl, unsubstituted or substituted heteroalicyclyl;
R₆, R₈ and R₉ are each hydrogen;
R₇ is halogen, heteroalicyclyl optionally substituted with —NX₁ₐX_{1b} or —SX₁ₐ, wherein X₁ₐ and X_{1b} are independently selected from the group consisting of hydrogen, unsubstituted or substituted C₁ to C₁₂ alkyl, unsubstituted or substituted C₂ to C₁₂ alkenyl, unsubstituted and substituted heteroaryl; and,
R₁₀ is selected from the group consisting of oxygen, —NH or NX_{1d} where X_{1d} is selected from the group consisting of hydrogen, substituted or unsubstituted aryl, substituted or unsubstituted heteroaryl, unsubstituted or substituted C₁ to C₁₀ alkyl, unsubstituted or substituted C₂ to C₁₀ alkenyl, unsubstituted or substituted C₂ to C₁₀ alkynyl, C₃ to C₁₀ cycloalkyl and C₅ to C₁₀ cycloalkenyl.

An aspect of this invention relates to a compound of this invention wherein:
R₁ is selected from the group consisting of hydrogen, methyl, 2-propyl, 2-butyl, aminocarbonylethyl, 2-methylmercaptoethyl, phenyl, benzyl, cyclohexylmethyl, 4-methoxybenzyl, 4-chlorobenzyl, 3-indolylmethyl, 4-(trifluoroacetyl)aminobutyl;
R₂, R₃, R₅, R₆, R₈ and R₉ are each hydrogen; and,
R₄ is —C(=Z)NX₁ₐX_{1b} wherein:
Z is oxygen or sulfur and X₁ₐ and X_{1b} together with the nitrogen to which they are covalently bonded form a group selected from the group consisting of 1-pyrrolidino, 4-methyl-1-homopiperazino, 4-(4-fluorophenyl)-1-piperazino, 4-(2-hydroxyethoxyethyl)-1-piperazino, 4-(2-pyridyl)-1-piperazino, 4-hydroxy-1-piperidino, 4-amino-2,2,6,6-tetramethyl-1-piperidino, 3-ethoxycarbonyl-1-piperidino, 4-(4-methoxyphenyl )-3-methyl-1-piperazino, 4-aminocarbonyl-1-piperidino, heptamethyleneimino, 4-(2-furoyl)-1-piperazino, 4-(3-trifluoromethylphenyl )-1-piperazino, 3-acetamido-1-pyrrolidino, 4-ethoxycarbonyl-1-piperazino, 4-ethoxycarbonyl-1-piperidino and 4-thiomorpholino, or
X₁ₐ is hydrogen and X_{1b} is selected from the group consisting of hydrogen, (1-ethyl-2-pyrrolidinyl)methyl, 2-thiazolyl, 5-methoxycarbonylpentyl, 2-ethoxy-carbonylethyl, 3-(methylthio)phenyl, N-methyl-( 1-methyl-4-piperidino), 2-(pyridin-2-yl )ethyl, 2-hydroxyethyl, 3-(trifluoromethyl)benzyl, N,N-dimethylaminoethyl, 3-(2-oxo-1-pyrrolidino)propyl, 1-ethoxycarbonyl4-piperidino, pyridin-2-ylmethyl, bis(2-methoxyethyl), 2-acetylaminoethyl, 3-(methylthio)propyl, 2-(1-morpholino)ethyl, 5-indazolyl, cyclopropyl, N-ethyl-(pyridin-4-ylmethyl), cyclopentyl, cycloheptyl, pyridin-3-ylmethyl, 4-(trifluoromethyl)benzyl, 2-(thien-2-yl)ethyl, 3-(N-pyrrolidino)propyl or 3-(1-imidazolyl)propyl; and
R₇ is selected from the group consisting of cyclopropylamino, 2-(1-morpholino)ethylamino, piperazino, 2-methyl-4-(3-methylphenyl )-1-piperazino, 4-aminocarbonylpiperidino, 2-(pyridin-2-yl)ethylamino, 2-(N,N-dimethylamino)ethylamino, 3-(aminomethyl)benzylamino, (5-phenyl-1H-1,2,4-triazol-3-yl)thio, 3-(4-morpholino)propylamino, tetrahydrofurfurylamino, 4-(2,5-dimethylphenyl)-1-piperazino, hexamethyleneimino, N-methyl-2-(pyridin-2-yl)ethylamino, 2-(dimethylamino)ethylamino, 4-(aminomethyl)benzylamino, (3-carboxypyridin-6-yl)thio, 2-acetylaminoethylamino, 2-(ethoxycarbonyl)ethylamino, 4-(2,3-dimethylphenyl)-1-piperazino, 4-(2-pyridyl)-1-piperazino, 3-(2-pipecolino)propylamino, 2-aminoethylamino, cyclohexylamino, imidazol-2-ylthio, 4-ethoxycarbonyl-1-piperazino, 3-methylthiopropylamino, 4-(4-fluorophenyl)piperazino, 1-benzyl-3-pyrrolidinoamino, N-methyl-4-piperidylamino, 3-aminopropylamino, N-benzylmethylamino, (3,5-dimethyl-2,6-pyrimidin-2-yl)thio, 4-acetyl-1-piperazino, 2,3-dimethoxybenzylamino, 4-(3,4-dichlorophenyl )-1-piperazino, 3-ethoxycarbonyl-1-piperidino, pyridin-3-ylmethylamino, N-methyl-2-(diethylamino)ethylamino, N-methylphenethylamino, (5-methyl-1,3,4-thiadiazol-2-yl)thio, 8-amino-3,6-dioxaoctyamino, 3-acetamido-1-pyrrolidino, 4-benzyl-1-piperazino, 4-ethoxycarbonyl-1-piperazino, 2-piperadinoethylamino, 3-dimethylaminopropylamino, cycloheptylamino, (1 H-1,2,4-triazol-3-yl)thio, 4-ethoxycarbonylmethyl-1-piperazino, 4-(diethylamino)-2-butenylamino, 4-(4-nitrophenyl)-1-piperazino, 1-ethoxycarbonyl-4-piperidylamino, 1-benzyl-4-piperidylamino, N-methyl-3-(dimethylamino)propylamino, 4-(trifluoromethyl)benzylamino, (4-methyl-1,2,4-triazol-3-yl)thio, 2-ethoxyethylamino, tyramino, 4-(3-trifluoromethylphenyl)-1-piperazino, 1,3,3-trimethyl-6-aza-6-bicyclo(3,2,1)-octyl, 3,3′-bis(dimethylamino)dipropylamino, butylamino, 3-(trifluoromethyl)benzylamino, pyridin-2-ylthio, 4-(2-furoyl)-1-piperazino, cyclooctylamino, 4-(4-acetylphenyl)-1-piperazino, 4-(4-methylphenyl )-3-methyl-1-piperazino, 2-fluorophenethylamino, 3-fluorophenethylamino, 4-fluorobenzylamino, fluoro, morpholino, thiomorpholino, 4-(5-chloro-2-methylphenyl )-1 -piperazino, (1-ethyl-2-pyrrolidino)methylamino, 2,2,6,6-tetramethyl-4-piperidylamino, diethylamino and 3,3,5-trimethylcyclo-hexyamino.

An aspect of this invention relates to a compound of this invention wherein:
R₁ is selected from the group consisting of hydrogen, methyl, 2-propyl, 2-butyl, aminocarbonylethyl, 2-methylmercaptoethyl, phenyl, benzyl, cyclohexylmethyl, 4-methoxybenzyl, 4-chlorobenzyl, 3-indolylmethyl, 4-(trifluoroacetyl)aminobutyl and 3-guanidinopropyl;
R₂, R₃, R₄ and R₅ are independently selected from the group consisting of hydrogen, methyl, carboxy, bromo, fluoro, chloro and trifluoromethyl;
R₆, R₈ and R₉ are each hydrogen;
R₇ is selected from the group consisting of cyclopropylamino, 2-(1-morpholino)ethylamino, piperazino, 2-methyl-4-(3-methylphenyl)-1-piperazino, 4-aminocarbonylpiperidino, 2-(pyridin-2-yl)ethylamino, 2-(N,N-dimethylamino)ethylamino, 3-(aminomethyl)-benzylamino, (5-phenyl-1H-1,2,4-triazol-3-yl)thio, 3-(4-morpholino)-propylamino, tetrahydrofurfurylamino, 4-(2,5-dimethylphenyl )-1-piperazino, hexamethyleneimino, N-methyl-2-(pyridin-2-yl)ethylamino, 2-(dimethylamino)ethylamino, 4-(aminomethyl)benzylamino, (3-carboxypyridin-6-yl)thio, 2-acetylaminoethylamino, 2-(ethoxycarbonyl)-ethylamino, 4-(2,3-dimethylphenyl)-1-piperazino, 4-(2-pyridyl)-1-piperazino, 3-(2-pipecolino)propylamino, 2-aminoethylamino, cyclohexylamino, imidazol-2-ylthio, 4-ethoxycarbonyl-1-piperazino, 3-methylthiopropylamino, 4-(4-fluorophenyl)piperazino, 1-benzyl-3-pyrrolidinoamino, N-methyl4-piperidylamino, 3-aminopropylamino, N-benzylmethylamino, (3,5-dimethyl-2,6-pyrimidin-2-yl )thio, 4-acetyl-1-piperazino, 2,3-dimethoxybenzylamino, 4-(3,4-dichlorophenyl)-1-piperazino, 3-ethoxycarbonyl-1-piperidino, pyridin-3-ylmethylamino, N-methyl-2-(diethylamino)ethylamino, N-methylphenethylamino, (5-methyl-1,3,4-thiadiazol-2-yl)thio, 8-amino-3,6-dioxaoctyamino, 3-acetamido-1-pyrrolidino, 4-benzyl-1-piperazino, 4-ethoxycarbonyl-1-piperazino, 2-piperadino-ethylamino, 3-dimethylaminopropylamino, cycloheptylamino, (1H-1,2,4-triazol-3-yl)thio, 4-ethoxycarbonylmethyl-1-piperazino, 4-(diethylamino)-2-butenylamino, 4-(4-nitrophenyl)-1-piperazino, 1-ethoxycarbonyl-4-piperidylamino, 1-benzyl4-piperidylamino, N-methyl-3-(dimethylamino)propylamino, 4-(trifluoromethyl)benzylamino, (4-methyl-1,2,4-triazol-3-yl)thio, 2-ethoxyethylamino, tyramino, 4-(3-trifluoromethylphenyl)-1-piperazino, 1,3,3-trimethyl-6-aza-6-bicyclo(3,2,1)-octyl, 3,3′-bis(dimethylamino)dipropylamino, butylamino, 3-(trifluoromethyl )benzylamino, pyridin-2-ylthio, 4-(2-furoyl )-1-piperazino, cyclooctylamino, 4-(4-acetylphenyl)-1-piperazino, 4-(4-methylphenyl)-3-methyl-1-piperazino, 2-fluorophenethylamino, 3-fluorophenethylamino, 4-fluorobenzylamino, fluoro, morpholino, thiomorpholino, 4-(5-chloro-2-methylphenyl)-1-piperazino, (1-ethyl-2-pyrrolidino)methylamino, 2,2,6,6-tetramethyl-4-piperidylamino, diethylamino and 3,3,5-trimethylcyclo-hexyamino; and,
R₁₀ is selected from the group consisting or oxygen, —NH and —NX_{1d} wherein X_{1d} is selected from the group consisting of hydrogen, unsubstituted or substituted aryl, unsubstituted or substituted heteroaryl, unsubstituted or substituted C₁ to C₁₀ alkyl, unsubstituted or substituted C₂ to C₁₀ alkenyl, unsubstituted or substituted C₂ to C₁₀ alkynyl, C₃ to C₁₀ cycloalkyl and C₅ to C₁₀ cycloalkenyl.

An aspect of this invention relates a compound of this invention having a chemical structure selected from the group consisting of:

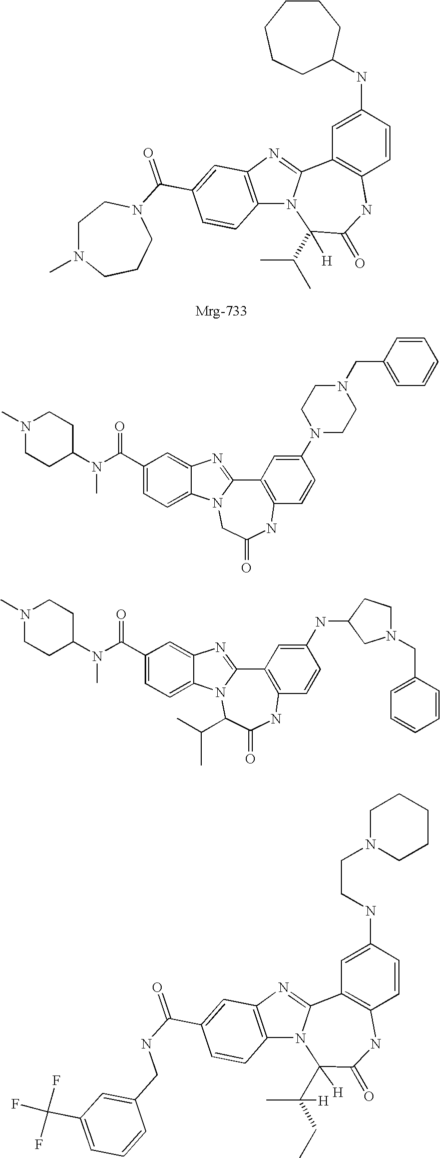

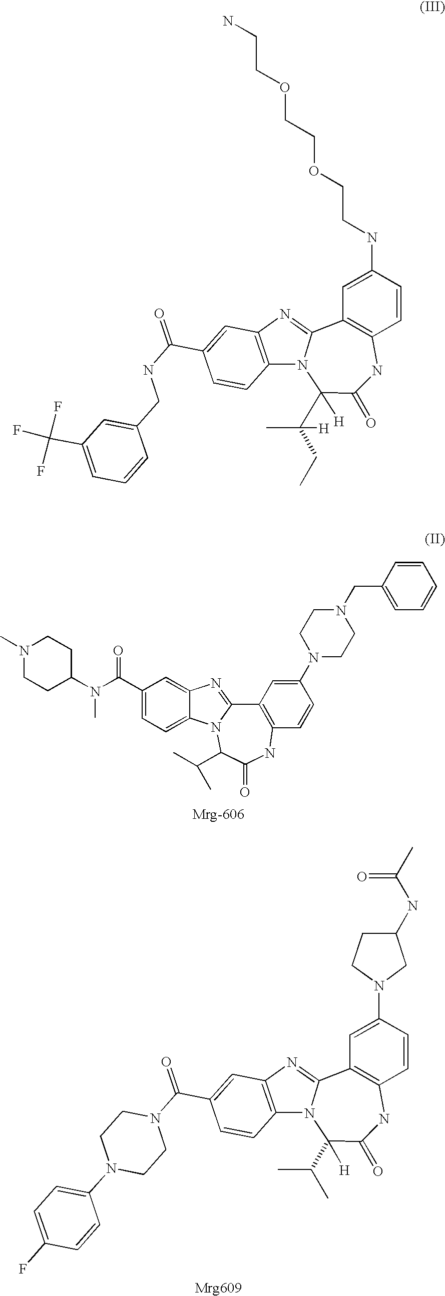

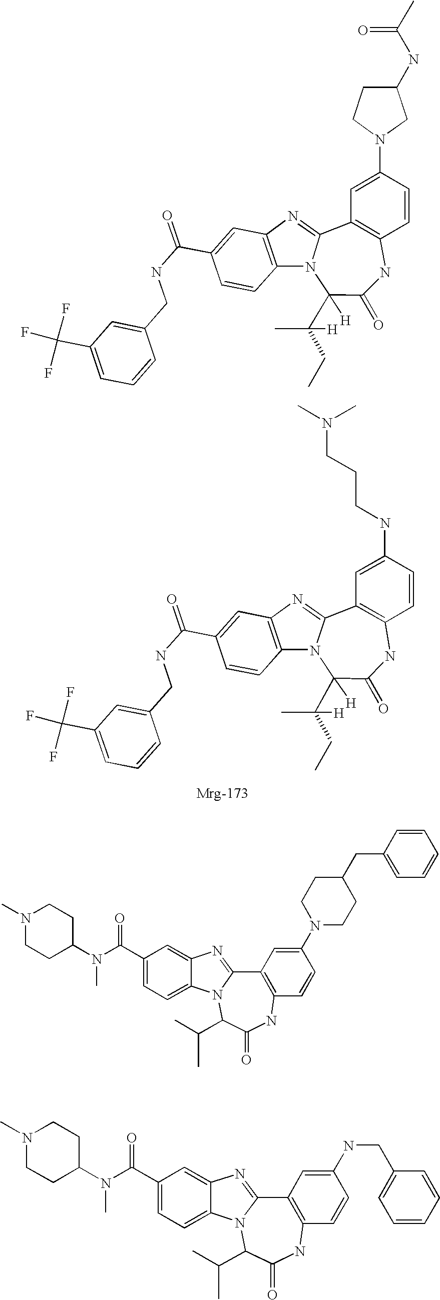

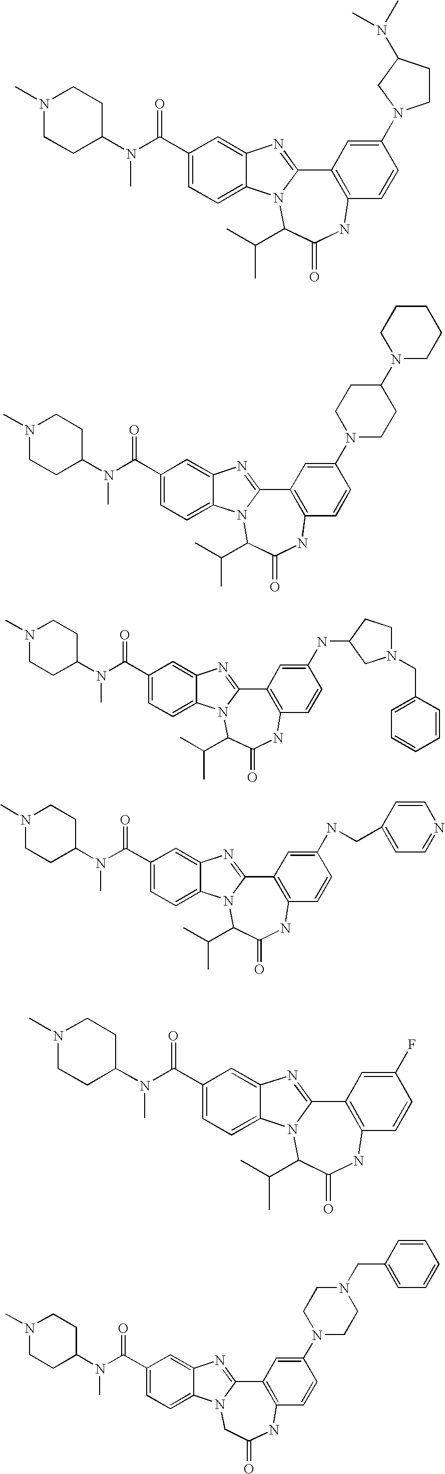

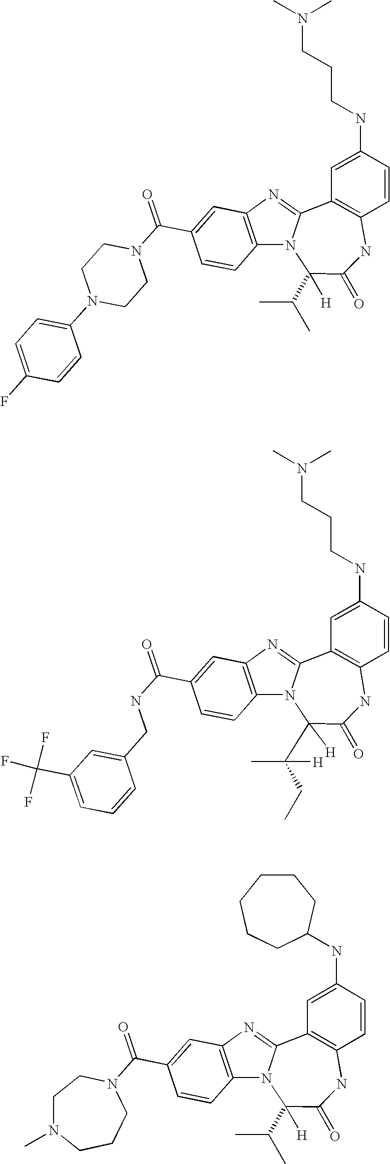

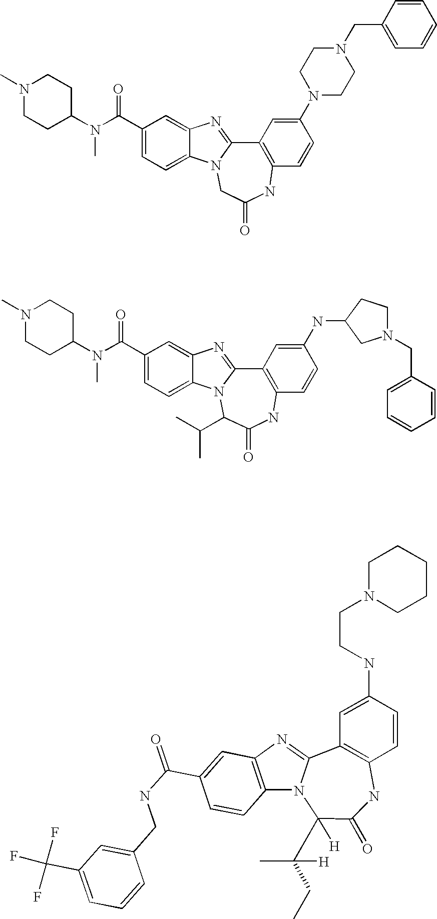

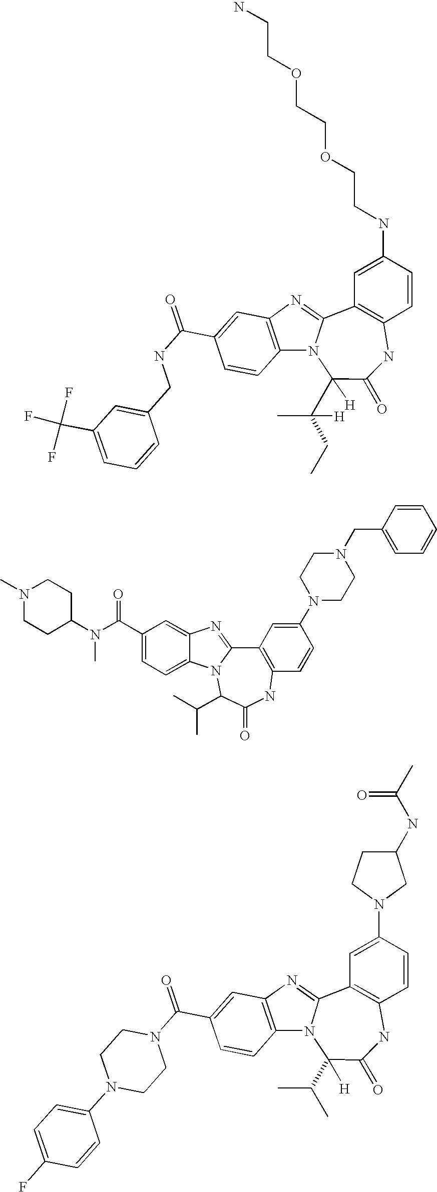

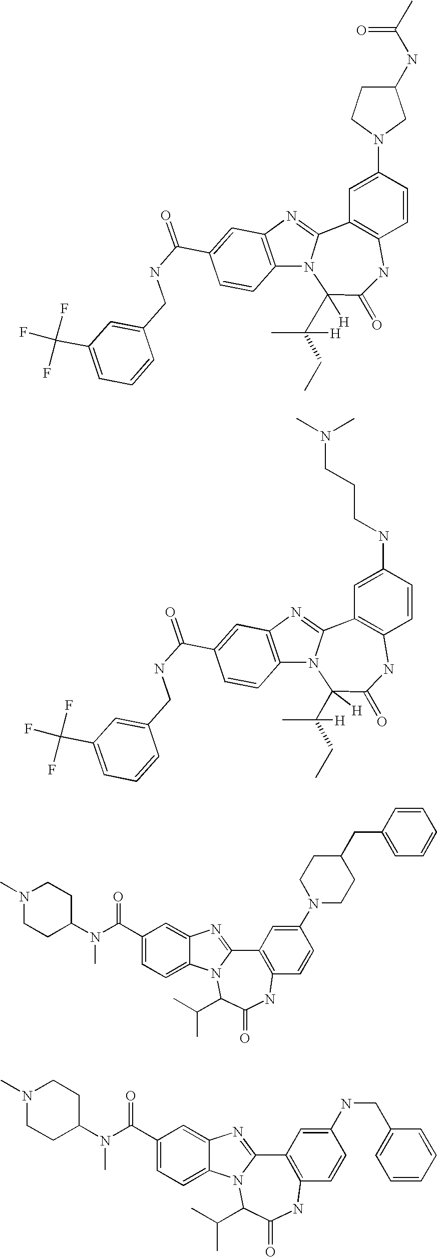

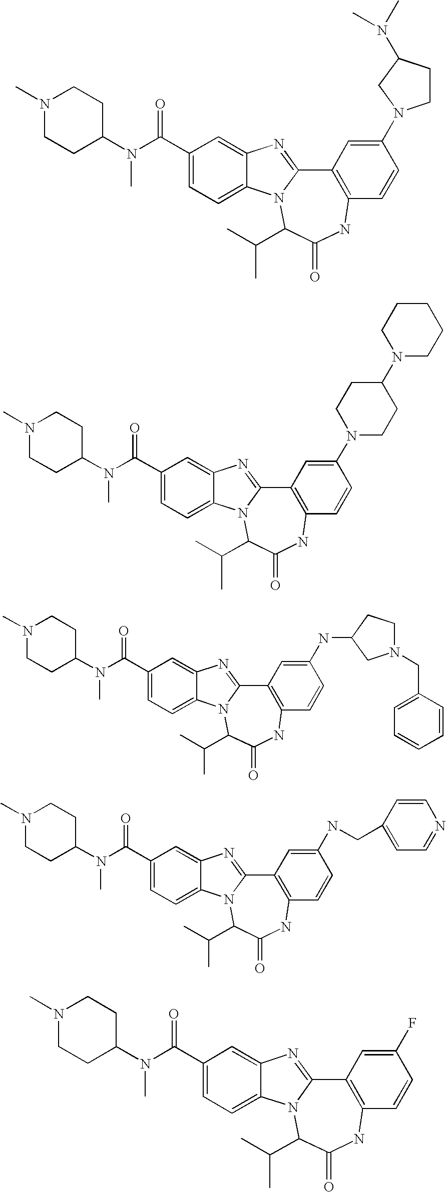

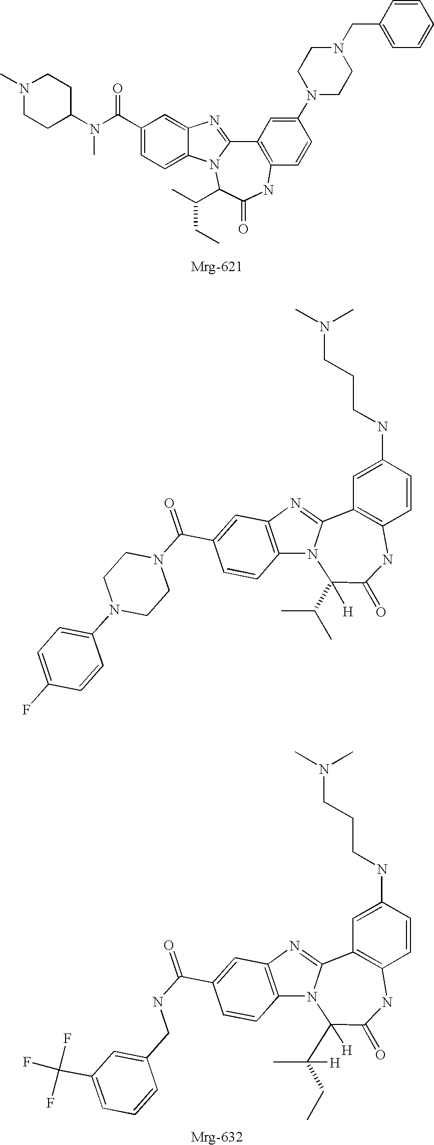

An aspect of this invention comprises a method of treating or preventing pain, comprising administering to a patient in need thereof an effective amount of at least one compound of this invention, or a salt or prodrug thereof.

An aspect of this invention relates to the use of a compound of this invention to treat or prevent pain in a patient in need thereof wherein the pain that is being treated or prevented is associated with diabetes, viral infection, irritable bowel syndrome, amputation, cancer, acute or chronic inflammation, arthritis, physical trauma or the side effects of other therapeutic drugs.

An aspect of this invention comprises a method of screening for a compound that modulates the activity of a human MrgX1, a simian MrgX1, a human MrgX2 or a simian MrgX2 receptor comprising contacting a test compound with a recombinant cell comprising a recombinant nucleic acid that expresses the human MrgX1, the simian MrgX1, the human MrgX2 or the simian MrgX2 receptor, provided that the cell does not contain an endogenous nucleic acid that expresses the functional receptor, and detecting changes in the activity of the receptor.

An aspect of this invention comprises the above method wherein the effect of the test compound on the cell or plurality of cells that express(es) the human MrgX1, the simian MrgX1, the human MrgX2 or the simian MrgX2 receptor is compared to its affect on the non-recombinant cell or plurality of cells. If the compound has no effect on the non-recombinant cell(s) but exhibits an effect on the recombinant cell(s), the compound is a specific agonist, inverse agonist or antagonist of the receptor.

An aspect of this invention comprises the above method wherein the recombinant nucleic acid is selected from the group consisting of:
a nucleic acid of SEQ ID NO:1;
a nucleic acid encoding the amino acid SEQ ID NO:2;
a nucleic acid of SEQ ID NO:3;
a nucleic acid encoding the amino acid SEQ ID NO:4;
a nucleic acid of SEQ ID NO:5;
a nucleic acid encoding the amino acid SEQ ID NO:6;
a nucleic acid of SEQ ID NO:7;
a nucleic acid encoding the amino acid SEQ ID NO:8;
a nucleic acid of SEQ ID NO:9;
a nucleic acid encoding the amino acid SEQ ID NO:10;
a nucleic acid of SEQ ID NO:11;
a nucleic acid encoding the amino acid of SEQ ID NO:12;
a nucleic acid of SEQ ID NO: 13;
a nucleic acid encoding the amino acid of SEQ ID NO:14;
a nucleic acid of SEQ ID NO 15; and,
a nucleic acid encoding the amino acid of SEQ ID NO:16.

An aspect of this invention relates to the above methods further comprising a nucleic acid that is at least 85% homologous with any one of SEQ ID NO:1, SEQ ID NO:3, SEQ ID NO:5, SEQ ID NO:7, SEQ ID NO:9, SEQ ID NO:11, SEQ ID NO. 13 and SEQ ID NO:15, and that encodes an amino acid sequence that exhibits substantially the same activity as the human MrgX1, the simian MrgX1, the human, MrgX2 or the simian MrgX2 receptor encoded by the aforementioned nucleic acid sequences.

### DETAILED DESCRIPTION

Brief Description of the Tables

TABLE 1 shows the selective activation of human MrgX1, simian MrgX1, human MrgX2 and simian MrgX2 receptor by compounds of this invention.

TABLE 2 shows that compounds of this invention are active at human MrgX1, simian MrgX1, human MrgX2 and simian MrgX2 receptor as indicated by their ability to stimulate intracellular calcium mobilization.

TABLE 3 shows that compounds of this invention are active at human MrgX1 or MrgX2 receptor as indicated by their ability to stimulate inositol phosphate hydrolysis.

### BRIEF DESCRIPTION OF THE FIGURES

FIG. 1 is a graphic representation of the selective activation of human MrgX1, simian MrgX1, human MrgX2 and simian MrgX2 receptor by compounds of this invention.

FIG. 2 is a graphic representation of the activity of compounds of this invention at human MrgX1, simian MrgX1, human MrgX2 and simian MrgX2 receptor as indicated by their ability to stimulate intracellular calcium mobilization.

FIG. 3 is another graphic representation of the activity of compounds of this invention at human MrgX1, simian MrgX1, human MrgX2 and simian MrgX2 receptor as indicated by their ability to stimulate intracellular calcium mobilization.

FIG. 4 is a graphic representation of the activity at human MrgX1 or MrgX2 receptor of compounds of this invention as indicated by their ability to stimulate inositol phosphate hydrolysis.

### DEFINITIONS

As used in these definitions, any “R” group(s), such as R^{a}, R^{b}, etc., is(are) independently selected from the group consisting of hydrogen, alkyl, alkenyl, alkynyl, cycloalkyl, aryl, heteroaryl (bonded to the indicated group at a ring carbon atom) and heteroalicyclyl (likewise bonded to the indicated group at a ring carbon atom), as these groups are defined herein. If two “R” groups are covalently bonded to the same atom or to adjacent atoms, then they may be taken together, as that term is defined herein, to form a cycloalkyl, aryl, heteroaryl or heteroalicyclyl group.

As used herein, the phrase “taken together” when referring to two “R” groups means that the “R” groups are joined together to form a cycloalkyl, aryl, heteroaryl or heteroalicyclyl group. For example, without limitation, if R^{a} and R^{b} of an NR^{a}R^{b} group are indicated to be “taken together,” it means that they are covalently bonded to one another at their terminal atoms to form a ring:

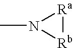

It is understood that, in any compound of this invention having one or more chiral centers, if an absolute stereochemistry is not expressly indicated, then each center may independently be R or S or a mixture thereof.

Whenever a group of this invention is described as being “optionally substituted” that group may be unsubstituted or substituted with one or more of the substituents described for that group. Likewise, when a group is described as being “unsubstituted or substituted,” if substituted, the substituent may be selected from the same group of substituents.

As used herein, “Cₘ to Cₙ” in which “m” and “n” are integers refers to the number of carbon atoms in an alkyl, alkenyl, alkynyl and the rings of cycloalkyl and cycloalkenyl group. That is, the alkyl, alkenyl or alkynyl can contain from “m” to “n”, inclusive, carbon atoms. If no “m” and “n” are designated with regard to an alkyl, alkenyl or alkynyl group herein, the broadest range described in these definitions is to be assumed. Thus “alkyl” alone means C₁-C₂₀ alkyl. A “C₁ to C₄ alkyl” group refers to all alkyl groups having from 1 to 4 carbons, that is, CH₃—, CH₃CH₂—, CH₃CH₂CH₂—, CH₃CH(CH₃), CH₃CH₂CH₂CH₂—, CH₃CH₂CH(CH₃)— and (CH₃)₃CH—, etc. With regard to cyclic compounds, “m” and “n” provide the number of possible carbon atoms in the ring.

As used herein, “alkyl” refers to a straight or branched chain fully saturated (no double or triple bonds) hydrocarbon (all carbon) group. An alkyl group of this invention may comprise from 1-20 carbon atoms, that is, “m”=1 and “n”=20, designated as a “C₁ to C₂₀ alkyl.” It is presently preferred that “m”=1 and “n”:=12 (C₁ to C₁₂ alkyl). It is presently more preferred that “m”=1 and “n”=6 (C₁ to C₆ alkyl). Examples of alkyl groups include, without limitation, methyl, ethyl, n-propyl, isopropyl, n-butyl, iso-butyl, sec-butyl, tert-butyl, amyl, tert-amyl, hexyl, heptyl, octyl, nonyl, decyl, undecyl and dodecyl.

An alkyl group of this invention may be substituted or unsubstituted. When substituted, the substituent group(s) is(are) one or more group(s) independently selected from cycloalkyl, aryl, heteroaryl, heteroalicyclyl, hydroxy, alkoxy, aryloxy, mercapto, alkylthio, arylthio, cyano, halo, oxo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, —NR^{a}R^{b}, protected hydroxyl, protected amino, protected carboxy and protected amido groups.

Examples of substituted alkyl groups include, without limitation, 2-oxo-prop-1-yl, 3-oxo-but-1-yl, cyanomethyl, nitromethyl, chloromethyl, hydroxymethyl, tetrahydropyranyloxymethyl, m-trityloxymethyl, propionyloxymethyl, aminomethyl, carboxymethyl, allyloxycarbonylmethyl, allyloxycarbonylaminomethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichlorobutyl, 2-aminopropyl, 1-chloroethyl, 2-chloroethyl, 1-bromoethyl, 2-chloroethyl, 1-fluoroethyl, 2-fluoroethyl, 1-iodoethyl, 2-iodoethyl, 1-chloropropyl, 2-chloropropyl, 3-chloropropyl, 1-bromopropyl, 2-bromopropyl, 3-bromopropyl, 1-fluoropropyl, 2-fluoropropyl, 3-fluoropropyl, 1-iodopropyl, 2-iodopropyl, 3-iodopropyl, 2-aminoethyl, 1-aminoethyl, N-benzoyl-2-aminoethyl, N-acetyl-2-aminoethyl, N-benzoyl-1-aminoethyl and N-acetyl-1-aminoethyl.

As used herein, “alkenyl” refers to an alkyl group that contains in the straight or branched hydrocarbon chain one or more double bonds. Examples of alkenyl groups include, without limitation, vinyl (CH₂═CH—), allyl (CH₃CH═CH₂—), 1-propenyl, 2-propenyl, 1-butenyl, 2-butenyl; 1-pentenyl, 2-pentenyl, 3-pentenyl, 4-pentenyl, 3-methyl-1-butenyl, and the various isomers of hexenyl, heptenyl, octenyl, nonenyl, decenyl undecenyl and dodecenyl.

An alkenyl group of this invention may be unsubstituted or substituted. When substituted, the substituent(s) may be selected from the same groups disclosed above with regard to alkyl group substitution. Examples of substituted alkenyl groups include, without limitation, styrenyl, 3-chloro-propen-1-yl, 3-chloro-buten-1-yl, 3-methoxy-propen-2-yl, 3-phenyl-buten-2-yl and 1-cyano-buten-3-yl.

As used herein, “alkynyl” refers to an alkyl group that contains in the straight or branched hydrocarbon chain one or more triple bonds.

An alkynyl group of this invention may be unsubstituted or substituted. When substituted, the substituent(s) may be selected from the same groups disclosed above with regard to alkyl group substitution.

As used herein, “cycloalkyl” refers to a completely saturated (no double bonds) hydrocarbon ring. Cycloalkyl groups of this invention may range from C₃ to C₁₀, preferably at present from C₃ to C₇. Examples of cycloalkyl groups include, without limitation, cyclopropyl, cyclobutyl, cyclopentyl, cyclohexyl and cycloheptyl.

As used herein, “cycloalkenyl” refers to a cycloalkyl group that contains one or more double bonds in the ring although, if there is more than one, they cannot form a fully delocalized pi-electron system in the ring (otherwise the group would be “aryl,” as defined herein). A cycloalkenyl of this invention may have from 5 to 10 carbon atoms in the ring, i.e., it may be C₅ to C₁₀, preferably at present C₅ to C₇. An cycloalkenyl group of this invention may unsubstituted or substituted. When substituted, the substituent(s) may be selected from the same groups disclosed above with regard to alkyl group substitution.

As used herein, “acyl” refers to an “RC(═O)O—” Examples of acyl groups include, without limitation, formyl, acetyl, propionyl, butyryl, pentanoyl, pivaloyl, hexanoyl, heptanoyl, octanoyl, nonanoyl, decanoyl, undecanoyl, dodecanoyl and benzoyl. Presently preferred acyl groups are acetyl and benzoyl.

An acyl group of this invention may be unsubstituted or substituted. When substituted, the substituent(s) may be selected from the same groups disclosed above with regard to alkyl group substitution. Example of substituted acyl groups include, without limitation, 4-phenylbutyroyl, 3-phenylbutyroyl, 3-phenylpropanoyl, 2-cyclohexanylacetyl, cyclohexanecarbonyl, 2-furanoyl and 3-dimethylaminobenzoyl.

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, naphthalene and azulene.

As used herein, “heteroaryl” refers to a ring or two or more fused rings that contain(s) one or more heteroatoms selected from the group consisting of nitrogen, oxygen and sulfur and that have a fully delocalized pi-electron system. Examples of heteroaryl groups include, but are not limited to, furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, triazine.

As used herein, “heteroalicyclic” or heteroalicyclyl” refers to a ring or one or more fused rings having in the ring system one or more heteroatoms independently selected from nitrogen, oxygen and sulphur. The rings may also contain one or more double bonds provided that they do not create a fully delocalized pi-electron system in the rings. Heteroalicyclyl groups of this invention may be unsubstituted or substituted. When substituted, the substituent(s) may be one or more groups independently selected from the group consisting of, without limitation, halogen, hydroxy, protected hydroxy, cyano, nitro, alkyl, alkoxy, acyl, acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N-alkylcarboxamide, protected N-alkylcarboxamide, N,N-dialkylcarboxamide, trifluoromethyl, N-alkylsulfonylamino and N-(phenylsulfonyl)amino. Presently preferred heteroalicyclyl groups include, without limitation, morpholino, piperidinyl, piperazinyl, 2-amino-imidazoyl, tetrahydrofurano, pyrrolo, tetrahydrothiophenyl, hexylmethyleneimino and heptylmethyleneimino.

As used herein, “phenylalkyl” refers to a phenyl ring covalently bonded to an alkyl group as defined herein. Examples, without limitation, of phenylalkyl groups include, without limitation, benzyl, 2-phenylethyl, 1-phenylpropyl, 4-phenylhexyl, 3-phenylamyl and 3-phenyl-2-methylpropyl. Presently preferred phenylalkyl groups are those wherein the phenyl group is covalently bonded to one of the presently preferred alkyl groups. A phenyl alkyl group of this invention may be unsubstituted or substituted. Examples of substituted phenylalkyl groups include, without limitation, 2-phenyl-1-chloroethyl, 2-(4-methoxyphenyl)ethyl, 4-(2,6-dihydroxy phenyl)hexyl, 2-(5-cyano-3-methoxyphenyl)pentyl, 3-(2,6-dimethylphenyl)propyl, 4-chloro-3-aminobenzyl, 6-(4-methoxyphenyl)-3-carboxy(n-hexyl), 5-(4-aminomethylphenyl)-3-(aminomethyl)pentyl and 5-phenyl-3-oxo-pent-1-yl.

As used herein, “heteroarylalkyl” and “heteroalicyclylalkyl” refer to a heteroaryl or a heteroalicyclyl group covalently bonded to an alkyl group, as defined herein. Examples of such groups include, without limitation, 2-pyridylethyl, 3-pyridylpropyl, 4-furylhexyl, 3-piperazylamyl and 3-morpholinylbutyl. Presently preferred heteroarylalkyl and heteroalicyclylalkyl groups are those in which a presently preferred heteroaryl or heteroalicyclyl group is covalently bonded to a presently preferred alkyl group as disclosed herein.

As used herein, “phenyl” refers to a 6-member aryl group. A phenyl group may be unsubstituted or substituted. When substituted the substituent(s) is/are one or more, preferably one or two, group(s) independently selected from the group consisting of halogen, hydroxy, protected hydroxy, cyano, nitro, alkyl, alkoxy, acyl, acyloxy, carboxy, protected carboxy, carboxymethyl, protected carboxymethyl, hydroxymethyl, protected hydroxymethyl, —NR^{a}R^{b} wherein R^{a} and R^{b} are as defined above but in addition R^{a} may be an amino protecting group as defined herein, carboxamide, protected carboxamide, N-alkylcarboxamide, protected N-alkylcarboxamide, N,N-dialkylcarboxamide, trifluoromethyl, N-alkylsulfonylamino, N-(phenylsulfonyl)amino and phenyl (resulting in the formation of a biphenyl group).

Examples of substituted phenyl groups include, without limitation, 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 and 4-fluorophenyl, 2, 3 or 4-hydroxyphenyl, 2,4-dihydroxyphenyl, the protected-hydroxy derivatives thereof, 2, 3 or 4-nitrophenyl; 2, 3 or 4-cyanophenyl; 2, 3 or 4-methylphenyl, 2,4-dimethylphenyl, 2, 3 or 4-(iso-propyl)phenyl, 2, 3 or 4-ethylphenyl, 2, 3 or 4-(n-propyl)phenyl, 2,6-dimethoxyphenyl, 2, 3 or 4-methoxyphenyl, 2, 3 or 4-ethoxyphenyl, 2, 3 or 4-(isopropoxy)phenyl, 2, 3 or 4-(t-butoxy)phenyl, 3-ethoxy-4-methoxyphenyl; 2, 3 or 4-trifluoromethylphenyl; 2, 3 or 4-carboxyphenyl or 2,4-di(protected carboxy)phenyl; 2, 3, or 4-(protected hydroxymethyl)phenyl or 3,4-di(hydroxymethyl)phenyl; 2, 3 or 4-(aminomethyl)phenyl or 2,4-(protected aminomethyl)phenyl; and 2, 3 or 4-(N-(methylsulfonylamino))phenyl.

As used herein, “phenylalkoxy” refers to a “phenylalkyl-O—” group with “phenyl” and “alkyl” as defined herein. A phenylalkoxy group of this invention may be substituted or unsubstituted on the phenyl ring, in the alkyl group or both. Examples of phenylalkoxy groups include, without limitation, 2-(4-hydroxyphenyl)ethoxy, 4-(4-methoxyphenyl)butoxy, (2R)-3-phenyl-2-amino-propoxy, (2S )-3-phenyl-2-amino-propoxy, 2-indanoxy, 6-phenyl-1-hexanoxy, cinnamyloxy, 2-phenyl-1-propoxy and 2,2-dimethyl-3-phenyl-1-propoxy.

As used herein, “halo” and “halogen” refer to the fluoro, chloro, bromo or iodo atoms. Presently preferred halogens are chloro and fluoro.

As used herein, “amino protecting group” refers to a group commonly employed to keep (i.e., to “block” or “protect”) an amino group from reacting with a reagent while it reacts with an intended target functional group of a molecule.

As used herein, a “protected carboxamide” refers to a carboxamide in which the nitrogen is substituted with an amino protecting group.

Examples of amino protecting groups include, without limitation, formyl (“For”), trityl, phthalimido, trichloroacetyl, chloroacetyl, bromoacetyl, iodoacetyl groups, t-butoxycarbonyl (“Boc”), 2-(4-biphenylyl)propyl-2-oxycarbonyl (“Bpoc”), 2-phenylpropyl-2-oxycarbonyl (“Poc”), 2-(4-xenyl)isopropoxycarbonyl, 1,1-diphenylethyl-1-oxycarbonyl, 1,1-diphenylpropyl-1-oxycarbonyl, 2-(3,5-dimethoxyphenyl)propyl-2-oxycarbonyl (“Ddz”), 2-(p-toluyl)propyl-2-oxycarbonyl, cyclopentanyloxycarbonyl, 1-methylcyclopentanyloxycarbonyl, cyclohexanyloxy-carbonyl, 1-methylcyclohexanyloxycarbonyl, 2-methylcyclohexanyloxycarbonyl, 2-(4-toluylsulfonyl)-ethoxycarbonyl, 2-(methylsulfonyl)ethoxycarbonyl, 2-(triphenylphosphino)-ethoxycarbonyl, 9-fluorenylmethoxycarbonyl (“Fmoc”), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyl-oxycarbonyl, 2,2, 2-trichloroethoxycarbonyl, 2-ethynyl-2-propoxycarbonyl, cyclopropyl-methoxycarbonyl, isobornyloxycarbonyl, 1-piperidyloxycarbonyl, benzyloxycarbonyl (“Cbz”), 4-phenylbenzyloxycarbonyl, 2-methylbenzyloxy-carbonyl, -2,4,5,-tetramethylbenzyloxycarbonyl (“Tmz”), 4-methoxybenzyloxy-carbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyl-oxycarbonyl, 4-bromobenzyloxycarbonyl, 3-bromobenzyloxycarbonyl, 4-nitrobenzyloxy-carbonyl, 4-cyanobenzyloxycarbonyl, 4-(decyloxy)benzyloxycarbonyl, benzoylmethylsulfonyl, dithiasuccinoyl (“Dts”),2-(nitro)phenylsulfenyl (“Nps”), and diphenyl-phosphine oxide. The species of amino-protecting group employed is not critical so long as the derivatized amino group is stable to the conditions of the subsequent reaction(s) and can be removed at the appropriate point without disrupting the remainder of the molecule. Presently preferred amino-protecting groups are Boc, Cbz and Fmoc. Descriptions of these and other amino-protecting groups may be found in T. W. Greene and P. G. M. Wuts, “Protective Groups in Organic Synthesis,” 2nd ed., John Wiley and Sons, New York, N.Y., 1991, Chapter 7, M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984.

As used herein, the term “carboxy protecting group” refers to a labile ester commonly used to block or protect a carboxylic acid while reactions are carried out on other functional groups on the compound. Examples of carboxy protecting groups include, without limitation, t-butyl, 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxytrityl, 4,4′,4″-trimethoxytrityl, 2-phenylpropyl, trimethylsilyl, t-butyldimethylsilyl, phenacyl, 2,2,2-trichloroethyl, -(trimethylsilyl)ethyl, -(di(n-butyl)methylsilyl )ethyl, p-toluenesulfonylethyl, 4-nitrobenzylsulfonylethyl, allyl, cinnamyl, and 1-(trimethylsilylmethyl)-propenyl. The ester employed is not critical so long as it is stable to the conditions of subsequent reaction(s) and can be removed at the appropriate point without disrupting the remainder of the molecule. Further examples of carboxy-protecting groups are found in E. Haslam, “Protective Groups in Organic Chemistry,” J. G. W. McOmie, Ed., Plenum Press, New York, N.Y., 1973, Chapter 5, and T. W. Greene and P. G. M. Wuts, “Protective Groups in Organic Synthesis,” 2nd ed., John Wiley and Sons, New York, N.Y., 1991, Chapter 5.

As used herein, a “hydroxyl protecting group” refers to a readily cleavable group that replaces the hydrogen of the hydroxyl group, such as, without limitation, tetrahydropyranyl, 2-methoxypropyl, 1-ethoxyethyl, methoxymethyl, 2-methoxyethoxymethyl, methylthiomethyl, t-butyl, t-amyl, trityl, 4-methoxytrityl, 4,4′-dimethoxytrityl, 4,4′,4″-trimethoxytrityl, benzyl, allyl, trimethylsilyl, (t-butyl)dimethylsilyl, and 2,2,2-trichloroethoxycarbonyl. The species of hydroxyl protecting groups is not critical so long as the derivatized hydroxyl group is stable to the conditions of subsequent reaction(s) and can be removed at the appropriate point without disrupting the remainder of the molecule. Further examples of hydroxy-protecting groups are described by C. B. Reese and E. Haslam, “Protective Groups in Organic Chemistry,” J. G. W. McOmie, Ed., Plenum Press, New York, N.Y., 1973, Chapters 3 and 4, respectively, and T. W. Greene and P. G. M. Wuts, “Protective Groups in Organic Synthesis,” 2nd ed., John Wiley and Sons, New York, N.Y., 1991, Chapters 2 and 3.

As used herein, “alkylthio” refers to an “alkyl-S—” group, with alkyl as defined above. Examples of alkylthio group include, without limitation, methylthio, ethylthio, n-propylthio, isopropylthio, n-butylthio and t-butylthio.

As used herein, “alkylsulfinyl” refers to an “alkyl-SO—” group, with alkyl as defined above. Examples of alkylsulfinyl groups include, without limitation, methylsulfinyl, ethylsulfinyl, n-propylsulfinyl, isopropylsulfinyl, n-butylsulfinyl and sec-butylsulfinyl.

As used herein, “alkylsulfonyl” refers to an “alkyl-SO₂—” group. Examples of alkylsulfonyl groups include, without limitation, methylsulfonyl, ethylsulfonyl, n-propylsulfonyl, isopropylsulfonyl, n-butylsulfonyl, and t-butylsulfonyl.

As used herein, “phenylthio,” “phenylsulfinyl,” and “phenylsulfonyl” refer to a “phenyl-S—,” “phenyl-SO—,” and “phenyl-SO₂—” group, phenyl as defined herein.

As used herein, “alkylaminocarbonyl” refers to an “alkylNHC(═O)—” group, with alkyl as defined herein. Examples of alkylaminocarbonyl groups include, without limitation, methylaminocarbonyl, ethylaminocarbonyl, propylaminocarbonyl and butylaminocarbonyl. Examples of substituted alkylaminocarbonyl include,without limitation, methoxymethyl-aminocarbonyl, 2-chloroethylaminocarbonyl, 2-oxopropylaminocarbonyl and 4-phenylbutylaminocarbonyl.

As used herein, “alkoxycarbonyl” refers to an “alkyl-OC(═O)—” group, with alkyl as defined above.

As used herein, “phenylaminocarbonyl” refers to a “phenyl-NHC(═O)—” group, with phenyl as defined above. Examples of substituted phenylaminocarbonyl groups include, without limitation, 2-chlorophenyl-aminocarbonyl, 3-chlorophenylaminocarbonyl, 2-nitorphenylaminocarbonyl, 4-biphenylaminocarbonyl, and 4-methoxyphenylaminocarbonyl.

As used herein, “alkylaminothiocarbonyl” refers to an “alkyl-NHC(═O)—” group, with alkyl as defined above. Examples of alkylaminothio-carbonyl groups include, without limitation, methylaminothiocarbonyl, ethylaminothiocarbonyl, propylaminothiocarbonyl and butylaminothiocarbonyl.

Examples of alkyl-substituted alkylaminothiocarbonyl groups include, without limitation, methoxymethylaminothiocarbonyl, 2-chloroethylaminothiocarbonyl, 2-oxopropylaminothiocarbonyl and 4-phenylbutylaminothiocarbonyl.

As used herein, “phenylaminothiocarbonyl” refers to a “phenyl-NHC(═S)—” group, with phenyl as defined above. Examples of phenylaminothiocarbonyl groups include, without limitation, 2-chlorophenylaminothiocarbonyl, 3-chlorophenylaminothiocarbonyl, 2-nitrophenylaminothiocarbonyl, 4-biphenylaminothiocarbonyl and 4-methoxyphenylaminothiocarbonyl.

As used herein, “carbamoyl” refers to an “—NCO—” group.

As used herein, “hydroxyl” refers to an “—OH” group.

As used herein, “cyano” refers to a “—C≡” group.

As used herein, “nitro” refers to an “—NO₂” group.

An “O-carboxy” group refers to a “RC(═O)O—”0 group with R as defined above.

A “C-carboxy” group refers to a “—C(═O)OR” group with R as defined above.

An “acetyl” group refers to a CH₃C(═O)— group.

A “trihalomethanesulfonyl” group refers to an “X₃CSO₂—” group wherein X is a halogen.

An “isocyanato” group refers to an “—NCO” group.

A “thiocyanato” group refers to a “—CNS” group.

An “isothiocyanato” group refers to an “—NCS” group.

A “sulfinyl” group refers to an “—S(═O)—R” group with R as defined above.

An “S-sulfonamido” group refers to a “—SO₂NR” group with R as defined above.

An “N-sulfonamido” group refers to a “RSO₂NH—” group with R as defined above.

A “trihalomethanesulfonamido” group refers to an “X₃CSO₂NR—” group with X as halogen and R as defined above.

An “O-carbamyl” group refers to a “—OC(═O)—NR” group with R as defined above.

An “N-carbamyl” group refers to an “ROC(═O)NH—” group with R as defined above.

An “O-thiocarbamyl” group refers to a “—OC(═S)—NR” group with R as defined above.

An “N-thiocarbamyl” group refers to an “ROC(═S)NH—” group with R as defined above.

A “C-amido” group refers to a “—C(═O)—NR^{a}R^{b} group with R^{a} and R^{b} as defined above.

An “N-amido” group refers to a RC(═O)NH— group with R as defined above.

The term “perhaloalkyl” refers to an alkyl group in which all the hydrogen atoms are replaced by halogen atoms.

As used herein, an “ester” refers to a “—C(O)OR^{a}” group with R^{a} as defined herein.

As used herein, an “amide” refers to a “—C(O)NR^{a}R^{b}” group with R^{a} and R^{b} as defined herein.

Any unsubstituted or monosubstituted amine group on a compound herein can be converted to an amide, any hydroxyl group can be converted to an ester and any carboxyl group can be converted to either an amide or ester using techniques well-known to those skilled in the art (see, for example, Greene and Wuts, Protective Groups in Organic Synthesis, 3^{rd} Ed., John Wiley & Sons, New York, N.Y., 1999). Compounds containing any such converted hydroxyl, amino and/or carboxylic acid groups are within the scope of this invention.

As used herein, an “ether” refers to an “—C—O—C—” group wherein either or both carbons may independently be part of an alkyl, alkenyl, alkynyl, aryl, heteroaryl or heteroalicyclyl group.

As used herein, a “halogenated ether” refers to an ether in which the groups to either side of the oxygen are both alkyl substituted with halogen.

As used herein, “amino acid” refers to any one of the twenty naturally-occurring L-amino acids, to their non-natural D-enantiomers, to non-naturally occurring amino acids such as, without limitation, norleucine (“Nle”), norvaline (“Nva”), L- or D-naphthalanine, ornithine (“Orn”), homoarginine (homoArg) and to other amino acids well-known in the peptide art such as those described in M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill.

Amino acids are referred to herein by their full chemical names or by their three letter codes, which are well-known to those skilled in the art. Unless the chirality of an amino acid is specifically designated or the amino acid is expressly stated to be a naturally occurring (i.e., L-) amino acid, the amino acid may be D or L or a racemic mixture of the two.

As used herein, a “functionalized resin” refers to any resin to which functional groups have been appended. Such functionalized resins are well-known to those skilled in the art and include, without limitation, resins functionalized with amino, alkylhalo, formyl or hydroxy groups. Examples of functionalized resins which can serve as solid supports for immobilized solid phase synthesis are well-known in the art and include, without limitation, 4-methylbenzhydrylamine-copoly(styrene-1% divinylbenzene) (MBHA), 4-hydroxymethylphenoxymethyl-copoly(styrene-1% divinylbenzene), 4-oxymethyl-phenyl-acetamido-copoly(stryene-1% divinylbenzene) (Wang), 4-(oxymethyl)-phenylacetamido methyl (Pam), and Tentagel™, from Rapp Polymere Gmbh, trialkoxy-diphenyl-methyl ester-copoly(styrene-1% divinylbenzene) (RINK) all of which are commercially available. Other functionalized resins useful in the synthesis of the compounds of this invention will become apparent to those skilled in the art based on the disclosures herein. All such resins are within the scope of this invention.

As used herein, “pharmaceutically acceptable salt” refers to a salt of a compound that does not cause significant irritation to a patient to which it is administered and does not abrogate the biological activity and properties of the compound. Pharmaceutical salts can be obtained by reaction of a compound disclosed herein with an acid or base to form a salt. Basic salts include, without limitation, ammonium salt (NH₄⁺) salts; alkali metal, such as, without limitation, sodium or potassium, salts; alkaline earth, such as, without limitation, calcium or magnesium, salts; salts of organic bases such as, without limitation, dicyclohexylamine, N-methyl-D-glucamine, tris(hydroxymethyl)methylamine; and salts with the amino group of with amino acids such as, without limitation, arginine and lysine. Useful acid salts include, without limitation, hydrochlorides, hydrobromides, sulfates, nitrates, phosphates, methanesulfonates, ethanesulfonates, p-toluenesulfonates and salicylates.

As used herein, a “prodrug” refers to a compound that may not be pharmaceutically active but that is converted into an active drug in vivo. Prodrugs are often useful because they may be easier to administer than the parent drug. They may, for example, be bioavailable by oral administration whereas the parent drug is not. The prodrug may also have better solubility than the active parent drug in pharmaceutical compositions. An example, without limitation, of a prodrug would be a compound disclosed herein, which is administered as an ester (the “prodrug”) to facilitate absorption through a cell membrane where water solubility is detrimental to mobility but which then is metabolically hydrolyzed to a carboxylic acid (the active entity) once inside the cell where water-solubility is beneficial. A further example of a prodrug might be a short peptide (polyaminoacid) bonded to an acid group where the peptide is metabolized in vivo to reveal the active parent.

As used herein, to “modulate” the activity of an MrgX receptor means either to activate it, i.e., to increase its cellular function over the base level measured in the particular environment in which it is found, or deactivate it, i.e., decrease its cellular function to less than the measured base level in the environment in which it is found and/or render it unable to perform its cellular function at all even in the presence of a natural binding partner. A natural binding partner is an endogenous molecule that is an agonist for the receptor.

As used herein, to “detect” changes in the activity of an MrgX receptor refers to the process of analyzing the result of an experiment using whatever analytical techniques are best suited to the particular situation. In some cases simple visual observation may suffice, in other cases the use of a microscope, visual or UV light analyzer or specific bioassays may be required. The proper selection of analytical tools and techniques to detect changes in the activity of MrgX receptors are well-known and will be apparent to those skilled in the art based on the disclosures herein.

As used herein, an “agonist” refers to a compound that binds to a receptor to from a complex that elicits the full pharmacological response associated with that particular receptor.

As used herein, “partial agonist” refers to a compound that has an affinity for a receptor but, unlike a full agonist, when bound to the receptor it elicits only a small degree of the pharmacological response normally associated with the receptor even if a large fraction of receptors are occupied by the compound.

As used herein, “inverse agonist” refers to a compound that inhibits the constitutive activity of a receptor such that the compound is not technically an antagonist but, rather, is an agonist with negative instrinsic activity.

As used herein, “antagonist” refers to a compound that binds to a receptor to form a complex that does not give rise to any response, as if the receptor were unoccupied. An antagonist often bind essentially irreversibly to the receptor, effectively eliminating the activity of the receptor permanently or at least until the antagonist is metabolized or otherwise removed by biological process.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes; and, in particular, humans.

As used herein, a “patient” refers to a subject that is being treated by an M.D. or a D.V.M. to attempt to cure, or at least ameliorate the effects of, a particular disease or disorder or to prevent the disease or disorder from occurring in the first place.

As used herein, a “therapeutically effective amount” refers to an amount of a compound that elicits the desired biological or medicinal response in an subject.

As used herein, a “pharmaceutical composition” refers to a mixture of a compound of this invention with other chemical components such as diluents, carriers or other excipients. A pharmaceutical composition may facilitate administration of the compound to a subject. Many techniques of administering a compound exist are known in the art, such as, without limitation, orally, intramuscularly, intraocularly, intranasally, parenterally, intravenously and topically. Pharmaceutical compositions will generally be tailored to the specific intended route of adminstration.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent ” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

Synthesis

The following syntheses are provide by way of illustration only and are not intended, nor should they be construed, as limiting the scope of this invention in any manner whatsoever. Those skilled in the art will, based on the disclosures herein, recognize modifications to the illustrated synthetic routes as well as other synthetic routes to the compounds herein; all such routes are within the scope of this invention.

Schemes 1-3 provide general synthetic routes to the compounds disclosed herein. Schemes 1 and 2 depict syntheses of the compounds using solid phase chemistry and scheme 3 provides a synthetic approach to the compounds using solution chemistry. 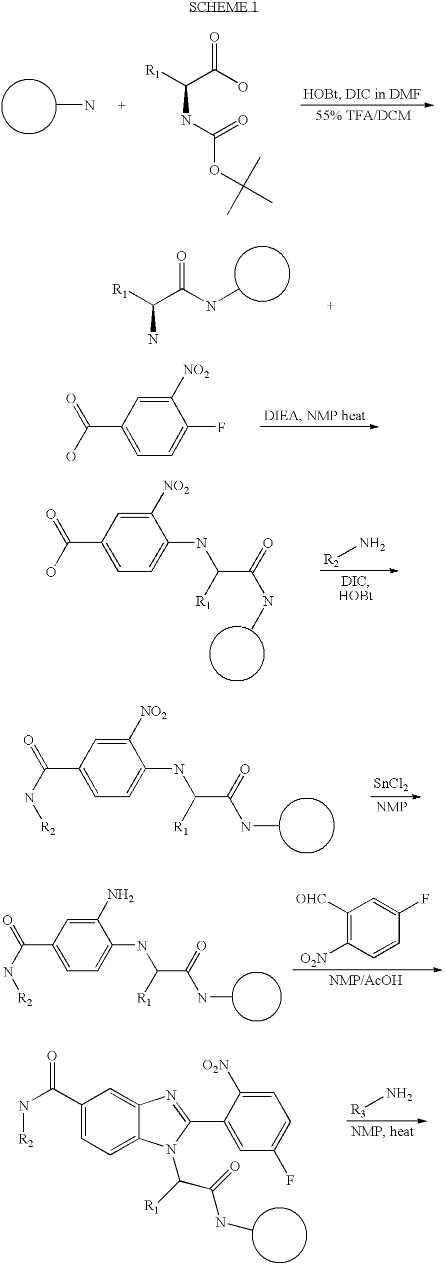 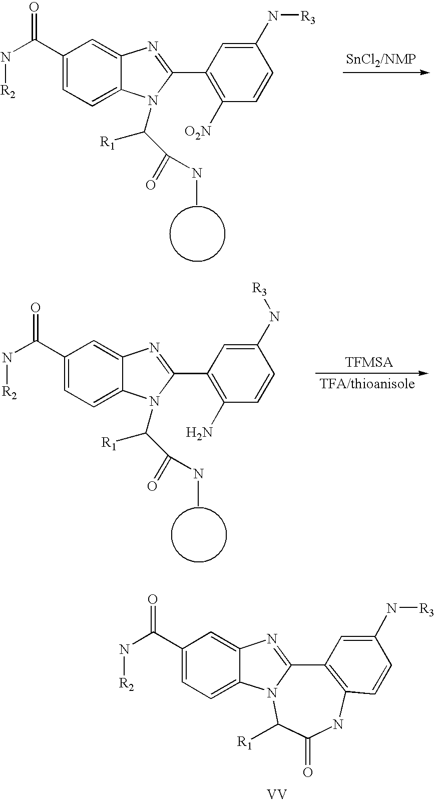 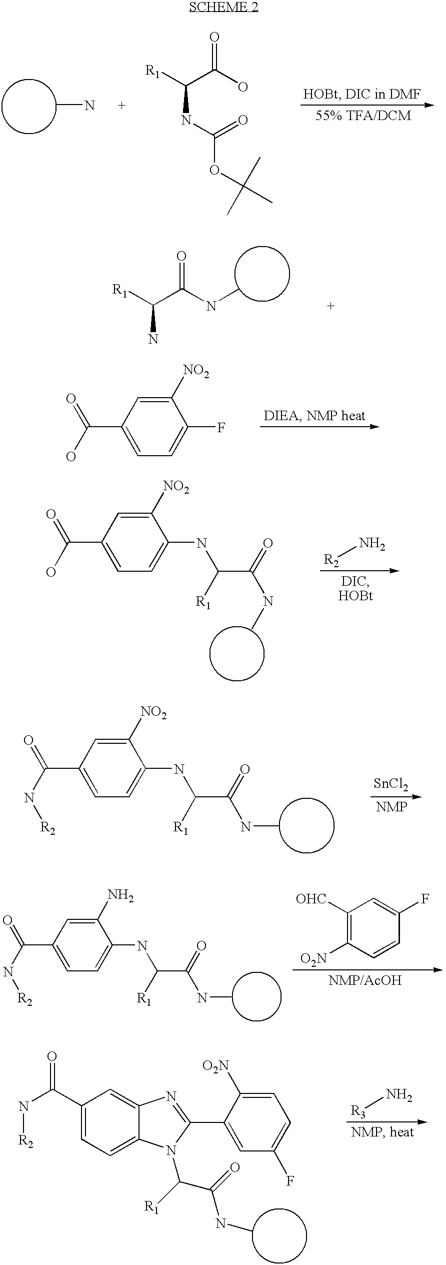 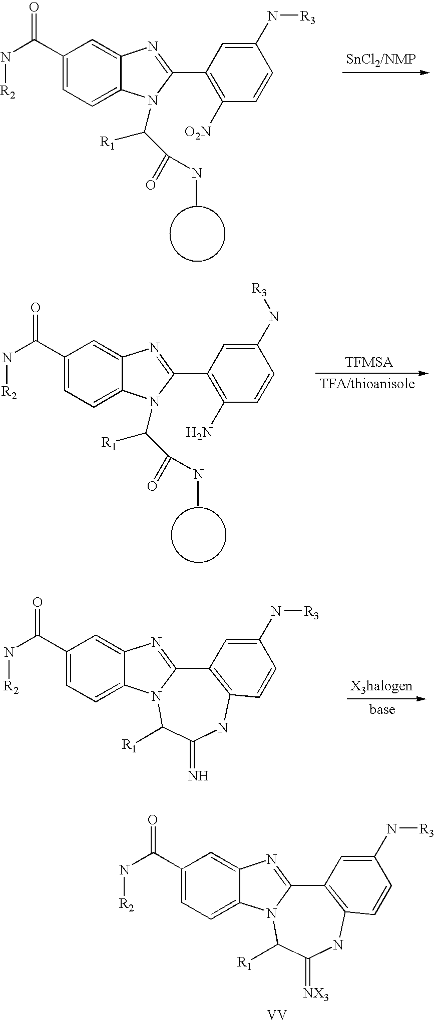 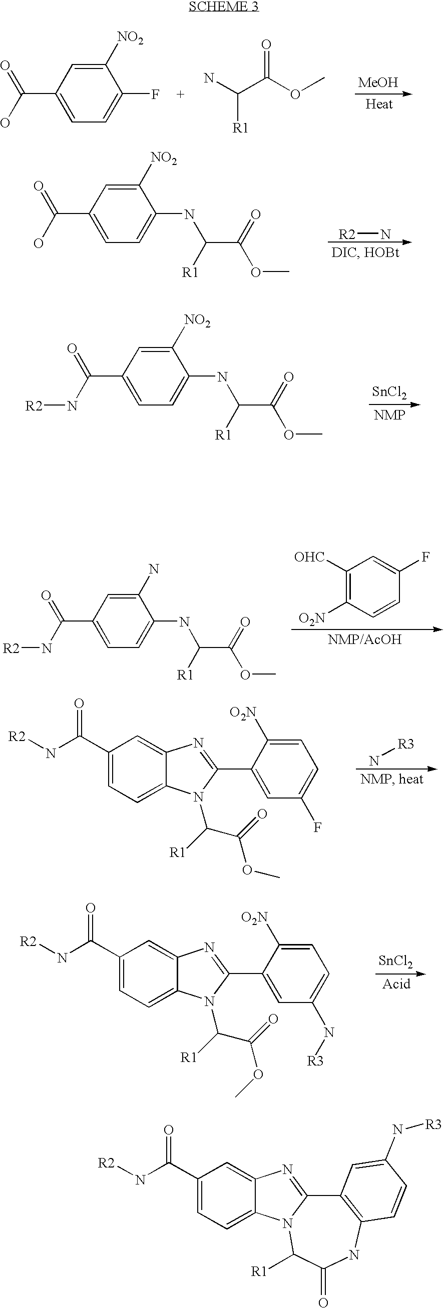
Pharmaceutics

The compounds of this invention can be administered to a human patient per se, or in a pharmaceutical composition where they are mixed with other active ingredients as, for example, in a combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, without limitation, include oral, rectal, transmucosal, or intestinal administration; parenteral delivery, including intramuscular, subcutaneous, intravenous, intramedullary injections, as well as intrathecal, direct intraventricular, intraperitoneal, intranasal, intraocular injections or as an aerosol inhalant.

Alternatively, one may administer the compound in a local rather than systemic manner, for example, via injection of the compound directly into the area of pain or inflammation, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions disclosed herein may be manufactured procedures well-known in the art, e.g., by means of conventional mixing, dissolving, granulating, dragee-making, levigating, emulsifying, encapsulating, entrapping or tabletting processes.

Pharmaceutical compositions for use in accordance with the present disclosure thus may be formulated in conventional manner using one or more pharmaceutically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents disclosed herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds disclosed herein is a co-solvent system comprising benzyl alcohol, a nonpolar surfactant, a water-miscible organic polymer, and an aqueous phase. A common co-solvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may be used.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations disclosed herein may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acids or base forms.

Pharmaceutical compositions suitable for use in the methods disclosed herein include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions disclosed herein can be chosen by the individual physician in view of the patient's condition. (See e.g., Fingl et al 1975, in “The Pharmacological Basis of Therapeutics”, Ch. 1 p. 1). Typically, the dose range of the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the methods disclosed herein will use those same dosages, or dosages that are between about 0.1% and 500%, or between about 25% and 250%, or between 50% and 100% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions disclosed herein or a pharmaceutically acceptable salt thereof calculated as the free base, the composition being administered 1 to 4 times per day. Alternatively the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

Dosage amount and interval may be adjusted individually to provide plasma levels of the active moiety, which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

### EXAMPLES

The following examples are provided by way of illustration only and are not intended, nor should they be construed, as limiting the scope of this invention in any manner whatsoever.

The following abbreviations are used:
MBHA: 4-methylbenzhydrylamine
DMF: dimethylformamide
HOBt: 1-hydroxybenzotriazole
NMP: N-methylpyrrolidone
Boc: tert-butoxycarbonyl
DIC: N,N′-diisopropylcarbodiimide
TFA: trifluoroacetic acid
DIEA: diisopropylethylamine
DCM dichloromethane
TFMSA: trifluoromethanesulfonic acid

### Example 1

Synthesis of Compound 2

Step 1

0.5 mg of MBHA resin (0.98 mmol/g) was placed in a polypropylene vial fitted with a filter. The polymer was swollen for 30 min in DCM, washed with 5% DIEA/DCM (2 times 10 mL) and then with DCM. A solution of Boc-valine (1.064 g, 4.9 mmol), DIC (0.927 g, 7.35 mmol), and HOBt (0.662 g, 4.9 mmol) in DMF (6 mL) was added. After shaking for 17 h, the resin was washed alternatively with DMF (6 mL) and MeOH (6 mL) for 3 cycles followed by DCM (6 mL) and MeOH (6 mL) for 3 cycles. The resin was dried in air for 1 h. A solution of 55% TFA/DCM (10 mL) was added and the mixture shaken at room temperature for 40 minutes. The solids were then washed with DCM (3×6 mL), 5% DIEA/DCM (3×6 mL) and MeOH (3×6 mL) and then air dried for 30 min.

Step 2

The resin from step1 was heated in a solution of 4-fluoro-3-nitrobenzoic acid (0.907 g, 4.9 mmol) and DIEA (0.633 g, 4.9 mmol) in N-methylpyrrolidinone (10 mL) at 70° C. for 24 h. The resin was washed alternatively with DMF (6 mL) and MeOH (6 mL) for 3 cycles followed by washing with DCM (3×6 mL) and MeOH (3×6 mL). The resin was then air-dried for 30 min.

Step 3

The resin from Step 2 was shaken with a solution of 1-Methyl-4-(4-methylamino)piperdirie (0.628 g, 4.9 mmol), DIC (0.927 g, 7.35 mmol) and HOBt (0.662 g, 4.9 mmol) in DMF (10 mL) for 24 hours. The resin was washed with DMF (3×6 mL) and MeOH (3×6 mL) followed by DCM (3×6 mL) and MeOH (3×6 mL). The resin was again air-dried for 30 min.

Step 4

The resin from step 3 was shaken with a 2.0 M solution of tin(II)chloride dihydrate in N-Methylpyrrolidinone (10 mL) for 24 h at room temperature. The resin was washed with DMF (3×6 mL), 5% DIEA/DCM (3×6 mL), MeOH, (3×6 mL), DMF (3×6 mL), MeOH (3×6 mL), DCM (3×6 mL) and MeOH (3×6 mL) and air dried for 30 min.

Step 5

The resin from step 4 was heated in a solution of 5-fluoro-2-nitrobenzaldehyde (0.828 g, 4.9 mmol) in N-methylpyrrolidinone (10 mL) and acetic acid (10 mL) at 70 ° C. for 72 h. The resin was washed alternatively with DMF (3×6 mL) and MeOH (3×6 mL) followed by washing with DCM (3×6 mL) and MeOH (3×6 mL). The resin was air dried in 30 min.

Step 6

The resin from step 5 was heated with 1-Benzylpiperazine (0.864 g, 4.9 mmol) in N-methylpyrrolidinone (10 mL ) at 75° C. for 72 h. The resin was washed with DMF (3×6 mL), MeOH, (3×6 mL), DMF (3×6 mL), MeOH (3×6 mL), DCM (3×6 mL) and MeOH (3×6 mL) and air dried for 30 min.

Step 7

The resin from step 6 was shaken with a 2.0 M solution of tin(II)chloride dihydrate in N-Methylpyrrolidinone (10 mL) for 24 h at room temperature. The resin was washed with DMF (3×6 mL), MeOH, (3×6 mL), DMF (3×6 mL), MeOH (3×6 mL), DCM (3×6 mL) and MeOH (3×6 mL) and air dried for 30 min.

Step 8

To the resin from step 7, thioanisole (1.250 mL) and TFA (12.5 mL) were added and the mixture shaken for 10 min. at rt. Then TFMSA (1.250 mL) was added and the mixture was shaken for an additional 2½ h. at rt. Ether (15 mL) was added and the precipitate that formed was washed with ether (4×15 mL) and dried.

### Example 2

Synthesis of Compound 3

Step 1

To a solution of isoleucine methyl ester (4.0 g, 22.02 mmol) and DIEA (2.82 g, 22.02 mmol) in methanol (20 mL) 4-fluoro-2-nitrobenzoic acid (1.02 g, 5.50 mmol) was added. The mixture was stirred at 65° C. for 48 h and then evaporated to dryness, dissolved in ethyl acetate and washed with water three times and then with a saturated NaCl solution. The organic layer was dried over Mg₂SO₄ and the course of the reaction verified by LC-MS.

Step 2

To a solution of the product of step 1, (2.34 g, 7.57 mmol), 3-(trifluoromethyl)benzylamine (1.32 g, 7.57 mmol), HOBt (1.22 g, 9.08 mmol), and DIC (1.15 g, 9.08 mmol) were added after cooling to 0° C. The mixture was stirred at rt for 48 h. The product was purified on silica gel using 5% methanol in DCM as eluent.

Step 3

Na₂S₂O₄ (3.73 g, 21.4 mmol) and Na₂CO₃ (2.25 g, 21.4 mmol) were dissolved in water (30 mL) and added to a solution of the compound from step 2 (2 g, 4.28 mmol) in ethanol (30 mL). After 1 h, the reaction was complete. The mixture was evaporated to dryness and extracted with ethyl acetate and water. The organic phase was separated, evaporated to dryness and used as is in the next step

Step 4

The product of step 3 (1.86 g, 4.25 mmol) and 5-fluoro-2-nitrobenzaldehyde (0.72 g, 4.25 mmol) were dissolved in a solution of acetic acid/N-Methylpyrrolidinone (1:1, 20:20 mL) and stirred at 70° C. for 72 h. The resulting product was purified on silica gel using 5% methanol in DCM as eluent. The product was further purified by preparative TLC using 5% methanol in DCM as eluent.

Step 5

The product of step 4 (0.498 g, 0.84 mmol) was dissolved in N-Methylpyrrolidinone (3.5 mL) and 3-(Dimethylamino)propylamine (1.6 mL, 12.6 mmol) was added. The mixture was then placed in a microwave oven for 10 min. at 100° C. This compound was used as is in the next step.

Step 6

The compound of step 5 was dissolved in a 2 M solution of SnCl₂ in N-methyl pyrrolidinone. The mixture was then placed in a microwave oven for 10 min. at 100° C. The solvent was then evaporated and the residual material purified by preparative TLC using CHCl₃/MeOH/H₂O/NH₃ (87:12:1:1) as eluent. The identity of the product was confirmed by LC-MS and NMR.

### Example 3

Receptor Selection and Amplification Technology Assay

The functional receptor assay, Receptor Selection and Amplification Technology (R-SAT), was used to investigate the pharmacological properties of test compounds on MRGX1 or MRGX2 receptors. R-SAT is disclosed in U.S. Pat. Nos. 5,707,798, 5,912,132, and 5,955,281, each of which is incorporated by reference herein in its entirety, including any drawings.

Briefly, NIH3T3 cells were grown in 96-well tissue culture plates to 70-80% confluence. Cells were transfected for 16-20 h with plasmid DNAs using Polyfect (Qiagen Inc.) and the manufacturer's protocols. R-SATs were generally performed with 4 ng/well of receptor and 20 ng/well of β-galactosidase plasmid DNA. The human MrgX1, simian MrgX1, human MrgX2 and simian MrgX2 receptor genes were amplified by PCR from genomic DNA using oligodeoxynucleotide primers based on SEQ ID NO:1, SEQ ID NO:3, SEQ ID NO:5, SEQ ID NO:7, or SEQ ID NO:9. For large-scale transfections, cells were transfected for 16-20 h, then trypsinized and frozen in DMSO. Frozen cells were later thawed, plated at ˜10,000 cells per well of a 96 half-area well plate that contained test compound. With both methods, cells were then grown in a humidified atmosphere with 5% ambient CO₂ for five days. The medium was then removed from the plates and marker gene activity was measured by the addition of the β-galactosidase substrate o-nitrophenyl β-galactopyranoside (ONPG, in PBS with 0.5% NP-40). The resulting colorimetric reaction was measured in a spectrophotometric plate reader (Titertek Inc.) at 420 nm. All data were analyzed using the computer program XLFit (IDBSm). Efficacy is defined as the percent maximal activation compared to activation by a control compound (BAM22 in the case of human or simian MRGX1, STIA or Cortistatin-14 in the case of human or simian MRGX2). pEC₅₀ is the negative of log(EC₅₀), where EC₅₀ is the calculated molar concentration of test compound that produces 50% of maximum activation.

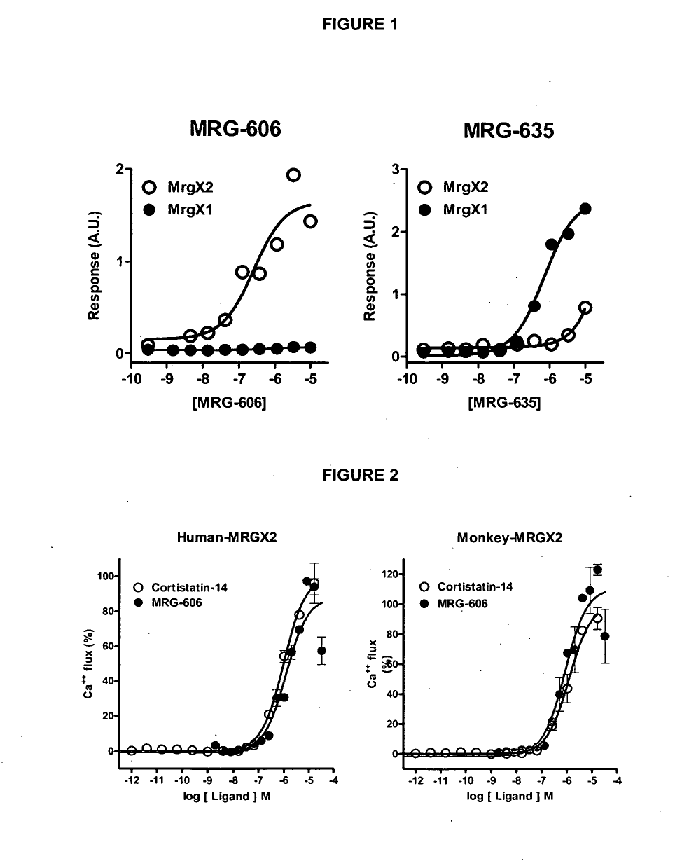

The experiments provided a molecular profile for each of the test compounds studied at the human MrgX1, simian MrgX1, human MrgX2 and simian MrgX2 receptors. As can be seen in Table 1, and FIG. 1, the compounds tested selectively activate human MrgX1, simian MrgX1, human MrgX2 and simian MrgX2 receptors.

TABLE 1

| | Generic | Human-X1 | | Monkey-X1 | | Human-X2 | | Monkey-X2 | |
|---|---|---|---|---|---|---|---|---|---|
| Compound | Structure | pEC50 | Eff(%) | pEC50 | Eff(%) | pEC50 | Eff(%) | pEC50 | Eff(%) |
| Mrg-606 | Formula I | — | nr | — | nr | 7.0 | 174 | 6.8 | 100 |
| Mrg-733 | Formula I | — | nr | 5.8 | 41 | 6.7 | 95 | 6.3 | 44 |
| Mrg-173 | Formula I | 7.4 | 124 | 6.4 | 144 | 6.5 | 98 | 5.8 | 64 |
| Mrg-609 | Formula I | 7.0 | 72 | 5.6 | 89 | — | nr | — | nr |

nr = no response up to 10 micromolar

Efficacy is reported relative to the ligands Bam22 for MrgX1 and STIA for MrgX2.

### Example 4

MrgX1 or MrgX2 Receptor Binding Assay

Using the following materials and methods, the ability of several of the compounds disclosed herein to bind to human MrgX1, simian MrgX1, human MrgX2 and simian MrgX2 receptors can be readily determined in a receptor binding assay.

1. Grow human MrgX1, simian MrgX1, human MrgX2 and simian MrgX2 receptor gene-transfected COS cells (other transfected cell lines that do not endogenously express human MrgX1, simian MrgX1, human MrgX2 or simian MrgX2 receptors may be substituted) in a suitable growth medium in 24-well culture plates.

2. Prepare a radio-labeled assay solution by mixing 245 μl of 0.25 nM [¹²⁵l] BAM22 or Cortistatin-14 working solution with 5 μl of the following (one per solution): 50 μM unlabeled BAM22 or Cortistatin-14 working solution, 0.25 nM [¹²⁵l] BAM22 or Cortistatin-14 working solution, HEPES buffer only, or serial dilutions of the test compound.

3. Aspirate the medium from the 24-well plates using a Pasteur pipet attached to a vacuum source. Do not wash cells.

4. Add 250 μl radiolabeled assay solution from step 2 to each assay well and the plates are incubated for 60 min at room temperature (˜22° C.) on an orbital shaker at low speed.

5. Terminate the incubation by aspirating the radioactive solution with a 24-well Brandel cell harvester. The wells are washed three times with 0.5 ml ice-cold HEPES buffer using the cell harvester.

6. Aspirate the solution from the wells with a micropipettor and transfer to 12×75 mm polystyrene test tubes. Analysis is then carried out using a gamma counter (Packard, Cobra Ill.).

7. Determine the specific binding and calculate the IC₅₀.

### Example 5

Determination of Changes in Cytosolic Calcium in Transfected HEK293 Cells

1. CHO—K1 cells transfected with human MrgX1, simian MrgX1, human MrgX2 and simian MrgX2 receptors or a control receptor at a density 1-3×10⁶ cells/ml are washed with phosphate—buffered saline.

2. Cells are loaded with 2 μM Fura-2 and analyzed with respect to the rise in intracellular calcium in the presence or absence of varying concentration of test compound.

3. The response is compared to that elicited by the application of the standard reference ligands BAM22 or Cortistatin-14 at 100 nM.

4. Intracellular free calcium concentrations are calculated using the formula:

$${[ {Ca}^{2 +} ]}_{i} = \frac{{K}_{d} ( F - {F}_{min} )}{{F}_{max} - F}$$

where K_{d} for Fura-2 is 224 nM; Fₘₐₓ is the fluorescence in the presence of 0.04% Triton-X100 and Fₘᵢₙ is the fluorescence obtained after the addition of 5 mM EGTA in 30 mM Tris-HCl, pH7.4.

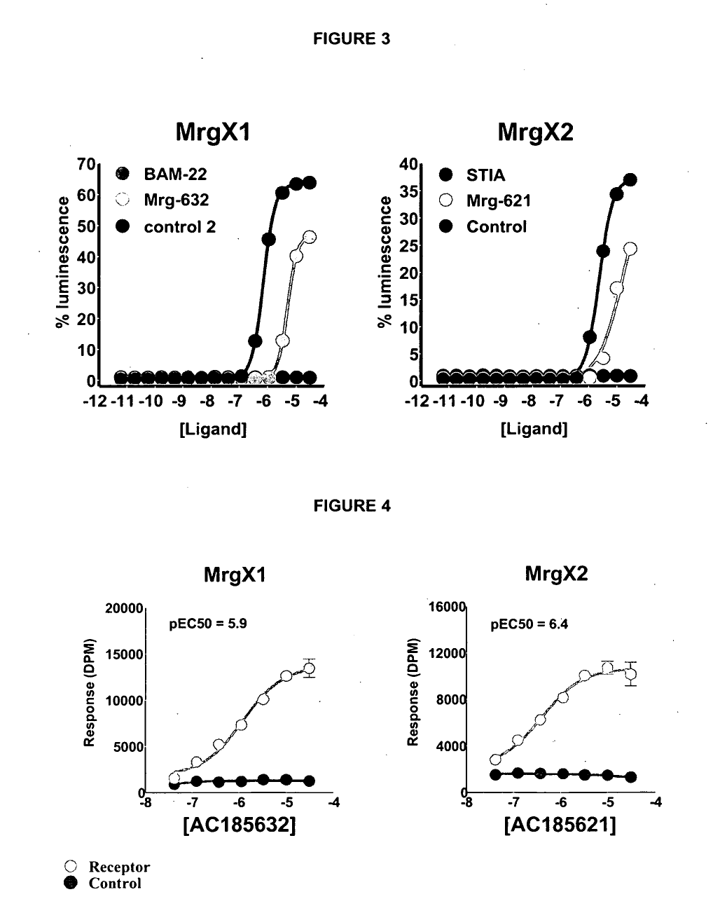

Table 2, FIG. 2 and FIG. 3 show that the compounds tested were each active at the human MrgX1, simian MrgX1, human MrgX2 and simian MrgX2 receptors as indicated by their ability to stimulate intracellular calcium mobilization.

TABLE 2

| | Generic | | |
|---|---|---|---|
| Compound | Structure | pEC₅₀ | % Efficacy |
| Mrg-632 | Formula I | 5.2 | 75 |
| Mrg-621 | Formula I | 5.2 | 65 |

### Example 6

Determination of Changes in Inositol Phosphates in Transfected TsA Cells

tsA cells (a transformed HEK293 cell line) were seeded at 10,000 cells/0.1 ml per well of 96 well plates at 37° C. in a humidified 5% CO₂ incubator in DMEM supplemented with 10% fetal calf serum, penicillin (100 units/ml) and streptomycin (100 mg/ml) and grown overnight. The cells were transfected with plasmid DNAs coding receptors, or G-protein helpers when needed, using PolyFect according to the same protocol used in the RSAT as described previously. At 18-20 h post-transfection, the medium was removed and the cells were labelled overnight with 2 uCi/ml myo-[2-3H] inositol (0.1 ml/well) freshly made in the culture medium. The medium was removed and the cells washed with Hank's Balanced Salt Solutions (HBSS) containing 1 mM CaCl₂, 1 mM MgCl₂, 20 mM LiCl and 0.1% BSA. The cells were then incubated with ligands for 45 min at 37° C. (0.1 ml/well) and the reaction was stopped by exchanging the buffer with 150 ul/well ice-cold 20 mM formic acid. 50ul/well 0.2M ammonium hydroxide was added and the plates were processed immediately or stored at −80° C.

To separate total [3H] inositol phosphates (IPs) ion-exchange chromatography columns were loaded with 200 ul of AG 1-X8 resin suspension (50% resin and 50% water) and the cell extracts were applied to the columns. The columns were washed with 1 ml of 40 mM ammonium hydroxide (pH9) and eluted [3H] IPs into 2 ml deep-well blocks with 0.4 ml 2M ammonium format0.1M formic acid. The column was washed with 0.6 ml water. The eluates were transferred into 7 ml scintillation vials and 5 ml liquid scintillation cocktail added. The wells were mixed well and the vials were left in the dark for at least 4 h and then counted on an LS 6500 Multi-purpose Scintillation Counter (3 min/vial). This procedure collects IP1, IP2 and IP3.

Table 3 and FIG. 4 show that the compounds tested were active at the human MRGX1 OR MRGX2 receptor in that they stimulated inositol phosphate hydrolysis.

TABLE 3

| | Generic | | |
|---|---|---|---|
| Compound | Structure | pEC₅₀ | % Efficacy |
| Mrg-632 | Formula I | 5.9 | 52 |
| Mrg-621 | Formula I | 6.4 | 87 |

### Example 7

Below are the DNA sequences encoding the human MrgX1 variant 1, human MrgX1 variant 2, simian MrgX1 variant 1, simian MrgX1 variant 2, human MrgX2, simian MRGX2 variant 1, simian MRGX2 variant 2, and simian MRGX2 variant 3 (SEQ ID NO:1, SEQ ID NO:3, SEQ ID NO:5, SEQ ID NO:7, SEQ ID NO:9, SEQ ID NO:11, SEQ ID NO:13 and SEQ ID NO:15). Below the DNA sequences are the polypeptide sequences for the human MrgX1 variant 1, human MrgX1 variant 2, simian MrgX1 variant 1, simian MrgX1 variant 2, human MrgX2, simian MRGX2 variant 1, simian MRGX2 variant 2, and simian MRGX2 variant 3 (SEQ ID NO:2, SEQ ID NO:4, SEQ ID NO:6, SEQ ID NO:8, SEQ ID NO:10, SEQ ID NO:12, SEQ ID NO:14 and SEQ ID NO:16). The variants described below were cloned from the genomic DNA of monkeys. The variants most likely represent polymorphic variations of these genes though they could represent separate, but highly homologous genes. Given the strong structural and functional similarities, any/all of these variants could mediate the actions of the compounds described herein, and any/all of the variants could be used in the methods for drug discovery described herein.

SEQ ID NO:1

atggatccaaccatctcaaccttggacacagaactgacaccaatcaacgg
aactgaggagactctttgctacaagcagaccttgagcctcacggtgctga
cgtgcatcgtttcccttgtcgggctgacaggaaacgcggttgtgctctgg
ctcctgggctgccgcatgcgcaggaacgccttctccatctacatcctcaa
cttggccgcagcagacttcctcttcctcagcggccgccttatatattccc
tgttaagcttcatcagtatcccccataccatctctaaaatcctctatcct
gtgatgatgttttcctactttgcaggcctgagctttctgagtgccgtgag
caccgagcgctgcctgtccgtcctgtggcccatctggtaccgctgccacc
gccccacacacctgtcagcggtggtgtgtgtcctgctctgggccctgtcc
ctgctgcggagcatcctggagtggatgttatgtggcttcctgttcagtgg
tgctgattctgcttggtgtcaaacatcagatttcatcacagtcgcgtggc
tgatttttttatgtgtggttctctgtgggtccagcctggtcctgctgatc
aggattctctgtggatcccggaagataccgctgaccaggctgtacgtgac
catcctgctcacagtactggtcttcctcctctgtggcctgccctttggca
ttcagtttttcctatttttatggatccacgtggacagggaagtcttattt
tgtcatgttcatctagtttctattttcctgtccgctcttaacagcagtgc
caaccccatcatttacttcttcgtgggctcctttaggcagcgtcaaaata
ggcagaacctgaagctggttctccagagggctctgcaggacgcgtctgag
gtggatgaaggtggagggcagcttcctgaggaaatcctggagctgtcggg
aagcagattggagcagtga

SEQ ID NO:2

MDPTISTLDTELTPINGTEETLCYKQTLSLTVLTCIVSLVGLTGNAVVLW
LLGCRMRRNAFSIYILNLAAADFLFLSGRLIYSLLSFISIPHTISKILYP
VMMFSYFAGLSFLSAVSTERCLSVLWPIWYRCHRPTHLSAVVCVLLWALS
LLRSILEWMLCGFLFSGADSAWCQTSDFITVAWLIFLCVVLCGSSLVLLI
RILCGSRKIPLTRLYVTILLTVLVFLLCGLPFGIQFFLFLWIHVDREVLF
CHVHLVSIFLSALNSSANPIIYFFVGSFRQRQNRQNLKLVLQRALQDASE
VDEGGGQLPEEILELSGSRLEQ*

SEQ ID NO:3

atggatccaaccgtctcaaccttggacacagaactgacaccaatcaacgg
aactgaggagactctttgctacaagcagaccttgagcctcacggtgctga
cgtgcatcgtttcccttgtcgggctgacaggaaacgcagttgtgctctgg
ctcctgggctgccgcatgcgcaggaacgccttctccatctacatcctcaa
cttggccgcagcagacttcctcttcctcagcggccgccttatatattccc
tgttaagcttcatcagtatcccccataccatctctaaaatcctctatcct
gtgatgatgttttcctactttgcaggcctgagctttctgagtgccgtgag
caccgagcgctgcctgtccgtcctgtggcccatctggtaccgctgccacc
gccccacacacctgtcagcggtggtgtgtgtcctgctctgggccctgtcc
ctgctgcggagcatcctggaatggatgttatgtggcttcctgttcagtgg
tgctgattctgcttggtgtcaaacatcagatttcatcacagtcgcgtggc
tgatttttttatgtgtggttctctgtgggtccagcctggtcctgctgatc
aggattctctgtggatcccggaagataccgctgaccaggctgtacgtgac
catcctgctcacagtactggtcttcctcctctgtggcctgccctttggca
ttcagtttttcctatttttatggatccacgtggacagggaagtcttattt
tgtcatgttcatctagtttctattttcctgtccgctcttaacagcagtgc
caaccccatcatttacttcttcgtgggctcctttaggcagcgtcaaaata
ggcagaacctgaagctggttctccagagggatctgcaggacacgcctgag
gtggatgaaggtggatggtgccttcctcaggaaaccctggagctgtcggg
aagcagattcaggcagtga

SEQ ID NO:4

MDPTVSTLDTELTPINGTEETLCYKQTLSLTVLTCIVSLVGLTGNAVVLW
LLGCRMRRNAFSIYILNLAAADFLFLSGRLIYSLLSFISIPHTISKILYP
VMMFSYFAGLSFLSAVSTERCLSVLWPIWYRCHRPTHLSAVVCVLLWALS
LLRSILEWMLCGFLFSGADSAWCQTSDFITVAWLIFLCVVLCGSSLVLLI
RILCGSRKIPLTRLYVTILLTVLVFLLCGLPFGIQFFLFLWIHVDREVLF
CHVHLVSIFLSALNSSANPIIYFFVGSFRQRQNRQNLKLVLQRDLQDTPE
VDEGGWCLPQETLELSGSRFRQ*

SEQ ID NO:5

atggatccaaccgtcccagtcttggacacaaaactgacaccaatcaaccg
aactgaggcgactccttgctacaagcagaccttgagcttcatggggctga
cgtgcatcatttcccttgtcggactgacaggaaacgcggttgtgctctgg
ctcctgggcttccgcatgcacaagaacgccttctccatctacatcctcaa
cctgtccatggccgacttcctcttcctcagtggccgctttatatattccc
tgttaagcttcatcagtgtgccccaaaccatctctaaaatcctctatcct
gtgatgatgttttcctactttgcaggcctgagctttctgagcgccatgag
caccgagcgctgcctgtccgttctgtggcccatgtggtaccgctgccgcc
gccccacacacctgtcagtggtcctgtgtgtcctgctctgggtcctgtcc
ctgctgcggagcatcctggagtggatattctgtggcttcctgtttagtgg
tgcggatcctgtttggtgtcaaacatcggatttcatcacagtcgcatggc
tgatttttttatgtgtggttctctgtgtgtccagcctggtcctagtgatc
aggattctctgtggatcccggaagatgccgctgaccaggctgtacgtgac
catcctgctcacagtgctggtcttcctcctctgcggcctgcccttcggcg
ttcagtttttcctatttttatggatccacgtggatcggaaagtcttatat
tgtcatgttcatctagtttctatgttcctggccgctcttaacagcagtgc
caaccccatcatttacttcttcgtgggctcctttaggcagcgtcaaaata
ggcagaacctgaggctgattctccagagggctctgcaggacacgcctgag
gtggatgaaggtggagggcggctgcctgaggaaaccctggagctgtcggg
aagcaaattggagatctga

SEQ ID NO:6

MDPTVPVLDTKLTPINRTEATPCYKQTLSFMGLTCIISLVGLTGNAVVLW
LLGFRMHKNAFSIYILNLSMADFLFLSGRFIYSLLSFISVPQTISKILYP
VMMFSYFAGLSFLSAMSTERCLSVLWPMWYRCRRPTHLSVVLCVLLWVLS
LLRSILEWIFCGFLFSGADPVWCQTSDFITVAWLIFLCVVLCVSSLVLVI
RILCGSRKMPLTRLYVTILLTVLVFLLCGLPFGVQFFLFLWIHVDRKVLY
CHVHLVSMFLAALNSSANPIIYFFVGSFRQRQNRQNLRLILQRALQDTPE
VDEGGGRLPEETLELSGSKLEI*

SEQ ID NO:7

atggatccaaccatcccagccttggacacaaaactgacaccaatcaaccg
aactgaggcgactccttgctacaagcagaccttgagcttcatggggctga
cgtgcatcatttcccttgtcggactgacaggaaacgcggttgtgctctgg
ctcctgggcttccgcatgcacaagaacgccttctccatctacatcctcaa
cctgtccatggccgacttcctcttcctcagtggccgctttatatattccc
tgttaagcttcatcagtgtgccccaaaccatctctaaaatcctctatcct
gtgacgatgttttcctactttgcaggcctgagctttctgagcgccatgag
caccgagcgctgcctgtccgttctgtggcccatgtggtaccgctgccgcc
gccccacacacctgtcagtggtcctgtgtgtcctgctgtgggtcctgtcc
ctgctgcggagcatcctggagtggatgttctgtggcttcctgtttagtgg
tgcggatcctgtttggtgtcaaacatcggatttcatcacagtcgcatggc
tgatttttttatgtgtggttctctgtgtgtccagcctggtcctagtgatc
aggattctctgtggatcccggaagatgccgctgaccaggctgtacgtgac
catcctgctcacagtgctggtcttcctcctctgcggcctgcccttcggcg
ttcagtttttcctatttttctggatccacgtggattggaaagtcttatat
tgtcatgttcatctagtttctatgttcctggccgctcttaacagcagtgc
caaccccatcatttacttcttcgtgggctcctttaggcagcgtcaaaata
ggcagaacctgaggctggttctccagagggctctgcaggacacgcctgag
gtggatgaaggtggagggcggctgcctgaggaaaccctggagctgtcggg
aagcagattggagcagtga

SEQ ID NO:8

MDPTIPALDTKLTPINRTEATPCYKQTLSFMGLTCIISLVGLTGNAVVLW
LLGFRMHKNAFSIYILNLSMADFLFLSGRFIYSLLSFISVPQTISKILYP
VTMFSYFAGLSFLSAMSTERCLSVLWPMWYRCRRPTHLSVVLCVLLWVLS
LLRSILEWMFCGFLFSGADPVWCQTSDFITVAWLIFLCVVLCVSSLVLVI
RILCGSRKMPLTRLYVTILLTVLVFLLCGLPFGVQFFLFFWIHVDWKVLY
CHVHLVSMFLAALNSSANPIIYFFVGSFRQRQNRQNLRLVLQRALQDTPE
VDEGGGRLPEETLELSGSRLEQ*

SEQ ID NO:9

atggatccaaccaccccggcctggggaacagaaagtacaacagtgaatgg
aaatgaccaagcccttcttctgctttgtggcaaggagaccctgatcccgg
tcttcctgatccttttcattgccctggtcgggctggtaggaaacgggttt
gtgctctggctcctgggcttccgcatgcgcaggaacgccttctctgtcta
cgtcctcagcctggccggggccgacttcctcttcctctgcttccagatta
taaattgcctggtgtacctcagtaacttcttctgttccatctccatcaat
ttccctagcttcttcaccactgtgatgacctgtgcctaccttgcaggcct
gagcatgctgagcaccgtcagcaccgagcgctgcctgtccgtcctgtggc
ccatctggtatcgctgccgccgccccagacacctgtcagcggtcgtgtgt
gtcctgctctgggccctgtccctactgctgagcatcttggaagggaagtt
ctgtggcttcttatttagtgatggtgactctggttggtgtcagacatttg
atttcatcactgcagcgtggctgatttttttattcatggttctctgtggg
tccagtctggccctgctggtcaggatcctctgtggctccaggggtctgcc
actgaccaggctgtacctgaccatcctgctcacagtgctggtgttcctcc
tctgcggcctgccctttggcattcagtggttcctaatattatggatctgg
aaggattctgatgtcttattttgtcatattcatccagtttcagttgtcct
gtcatctcttaacagcagtgccaaccccatcatttacttcttcgtgggct
cttttaggaagcagtggcggctgcagcagccgatcctcaagctggctctc
cagagggctctgcaggacattgctgaggtggatcacagtgaaggatgctt
ccgtcagggcaccccggagatgtcgagaagcagtctggtgtag

SEQ ID NO:10

MDPTTPAWGTESTTVNGNDQALLLLCGKETLIPVFLILFIALVGLVGNGF
VLWLLGFRMRRNAFSVYVLSLAGADFLFLCFQIINCLVYLSNFFCSISIN
FPSFFTTVMTCAYLAGLSMLSTVSTERCLSVLWPIWYRCRRPRHLSAVVC
VLLWALSLLLSILEGKFCGFLFSDGDSGWCQTFDFITAAWLIFLFMVLCG
SSLALLVRILCGSRGLPLTRLYLTILLTVLVFLLCGLPFGIQWFLILWIW
KDSDVLFCHIHPVSVVLSSLNSSANPIIYFFVGSFRKQWRLQQPILKLAL
QRALQDIAEVDHSEGCFRQGTPEMSRSSLV*

SEQ ID NO:11

atggatccaaccaccccggcctggggaaccgaaagtacaacaatgaatgg
aaatgatcaagcccttcctctgctttgtggcaaggagaccatgatctcgg
tcttcctgatcctcttcattgccctggtagggctggtaggaaacgcgttt
gtgctctggctcctgggcttccgcatgcgcagaaacgccttctcggtcta
cgtcctcagcctggccggggccgacttcctcttcctctgcttccagatga
caaattgcctggcatacctcattaacttcttcggttccatctccatcaat
ttccctagcttcttcaccactgtgatgacctgtgcctaccttgcaggcct
gagcatgctaagcgccatcagcaccgagcgctgcctgtccgtcctgtggc
ccatctggtaccgctgccgccgccccagacacctgtcagcggtcatgtgt
gtcctgctctgggccctgtccctgctgctgagcatcttggaagggaagtt
ctgtggcttcttatttagtgatggtgactctggttggtgtcagacatttg
atttcatcacagcagcgtggctgatgtttttatttgtggttctctgtgga
tccagcctggccctgctggtcaggatcctctgtggctcccggggtctgcc
actgaccaggctgtacctgaccatcctgctcaccgtgctgatcttcctcc
tctgcggcctgcccttgggcattcagtggttcctaatattatggatctgg
aagagttctgatgtgttattttgtcatattcatccagtttcagttgtcct
gtcgtcttttaacagcagtgccaaccctatcatttacttcttcgtgggct
cctttaggaagcagtggcggctccggcagccgatcctcaagctggctctc
cagagggctctgcaggacactgctgaggtggatcacagtgaaggatgctt
cagtcagggcaccctggagatgtccagaagcagtctggtgtag

SEQ ID NO:12

MDPTTPAWGTESTTMNGNDQALPLLCGKETMISVFLILFIALVGLVGNAF
VLWLLGFRMRRNAFSVYVLSLAGADFLFLCFQMTNCLAYLINFFGSISNF
PSFFTTVMTCAYLAGLSMLSAISTERCLSVLWPIWYRCRRPRHLSAVMCV
LLWALSLLLSILEGKFCGFLFSDGDSGWCQTFDFITAAWLMFLFVVLCGS
SLALLVRILCGSRGLPLTRLYLTILLTVLIFLLCGLPLGIQWFLILWIWK
SSDVLFCHIHPVSVVLSSFNSSANPIIYFFVGSFRKQWRLRQPILKLALQ
RALQDTAEVDHSEGCFSQGTLEMSRSSLV*

SEQ ID NO:13

atggatccaaccaccccagcctggggaaccgaaagtacaacaatgaatgg
aaatgatcaagcccttcctctgctttgtggcaaggagaccatgatctcgg
tcttcctgatcctcttcattgccctggtagggctggtaggaaacgcgttt
gtgctctggctcctgggcttccgcatgcgcagaaacgccttctctgtcta
cgtcctcagcctggccggggccgacttcctcttcctctgcttccagatga
caagttgcctggcatacctcattaacttcttcggttccatctccatcaat
atccctagcttcttcactgtgatgacctgtgcctaccttgcaggcctgag
catgctaagcgccatcagcaccgagcgctgcctgtccgtcctgtggccca
tctggtaccgctgccgccgccccagacacctgtcagcggtcatgtgtgtc
ctgctctgggccctgtccctgctgctgagcatcttggaagggaagttctg
tggcttcttatttagtgatgatgaccctggttggtgtcagacatttgatt
tcatcacagcagcgtggctgatgtttttatttgtggttctctgtggatcc
agcctggccctgctggtcaggatcctctgtggctcccggagtctgccact
gaccaggctgtacctgaccatcctgctcaccgtgctgatcttcctcctct
gcggcctgcccttcggcattcagtggttcctaatattatggatctggaag
aattctgttgtgttattttgtcatattcatccaatttcagttgtcctgtc
gtcttttaacagcagtgccaaccctatcatttacttcttcgtgggctcct
ttaggaagcagtggcggctccggcagccgatcctcaagctggctctccag
agggctctgcaggacactgctgaggtggatcacagtgaaggatgcttcag
tcagggcaccctggagatgtccagaagcagtctggtgtag

SEQ ID NO:14

MDPTTPAWGTESTTMNGNDQALPLLCGKETMISVFLILFIALVGLVGNAF
VLWLLGFRMRRNAFSVYVLSLAGADFLFLCFQMTSCLAYLINFFGSISIN
IPSFFTVMTCAYLAGLSMLSAISTERCLSVLWPIWYRCRRPRHLSAVMCV
LLWALSLLLSILEGKLFCGFLFSDDDPGWCQTFDFITAAWLMFLFVVLCG
SSLALLVRILCGSRSLPLTRLYLTILLTVLIFLLCGLPFGIQWFLILWIW
KNSVVLFCHIHPISVVLSSFNSSANPIIYFFVGSFRKQWRLRQPILKLAL
QRALQDTAEVDHSEGCFSQGTLEMSRSSLV*

SEQ ID NO:15

atggatccaaccaccccagcctggggaaccgaaagtacaacaatgaatgg
aaatgatcaagcccttcctctgctttgtggcaaggagaccatgatctcgg
tcttcctgatcctcttcattgccctggtagggctggtaggaaacgcgttt
gtgctctggctcctgggcttccgcatgcgcagaaacgccttctctgtcta
cgtcctcagcctggccggggccgacttcctcttcctctgcttccagatga
caagttgcctggcatacctcattaacttcttcggttccatctccatcaat
atccctagcttcttcactgtgatgacctgtgcctaccttgcaggcctgag
catgctaagcgccatcagcaccgagcgctgcctgtccgtcctgtggccca
tctggtaccgctgccgccgccccagacacctgtcagcggtcatgtgtgtc
ctgctctgggccctgtccctgctgctgagcatcttggaagggaagttctg
tggcttcttatttagtgatgatgactctggttggtgtcagacatttgatt
tcatcacagcagcgtggctgatgtttttatttgtggttctctgtggatcc
agcctggccctgctggtcaggatcctctgtggctcccggagtctgccact
gaccaggctgtacctgaccatcctgctcaccgtgctgatcttcctcctct
gcggcctgcccttcggcattcagtggttcctaatattatggatctggaag
aattctgttgtgttattttgtcatattcatccaatttcagttgtcctgtc
gtcttttaacagcagtgccaaccctatcatttacttcttcgtgggctcct
ttaggaagcagtggcggctccggcagccgatcctcaagctggctctccag
agggctctgcaggacactgctgaggtggatcacagtgaaggatgcttcag
tcagggcaccctggagatgtccagaagcagtctggtgtag

SEQ ID NO:16

MDPTTPAWGTESTTMNGNDQALPLLCGKETMISVFLILFIALVGLVGNAF
VLWLLGFRMRRNAFSVYVLSLAGADFLFLCFQMTSCLAYLINFFGSISIN
IPSFFTVMTCAYLAGLSMLSAISTERCLSVLWPIWYRCRRPRHLSAVMCV
LLWALSLLLSILEGKFCGFLFSDDDSGWCQTFDFITAAWLMFLFVVLCGS
SLALLVRILCGSRSLPLTRLYLTILLTVLIFLLCGLPFGIQWFLILWIWK
NSVVLFCHIHPISVVLSSFNSSANPIIYFFVGSFRKQWRLRQPILKLALQ
RALQDTAEVDHSEGCFSQGTLEMSRSSLV*

What is claimed:

1. A compound having the chemical formula: 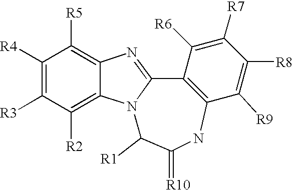
wherein:
R₁ is selected from the group consisting of hydrogen, unsubstituted or substituted alkyl, unsubstituted or substituted alkenyl, unsubstituted or substituted phenyl, unsubstituted or substituted naphthyl, unsubstituted or substituted phenylalkyl, unsubstituted or substituted heteroaryl, unsubstituted or substituted heteroalicyclyl, cyano, unsubstituted or substituted acyl, unsubstituted or substituted alkoxycarbonyl, unsubstituted or substituted alkylaminocarbonyl, unsubstituted or substituted phenylaminocarbonyl, unsubstituted or substituted alkylthio, unsubstituted or substituted alkylsulfonyl, unsubstituted or substituted alkylsulfinyl, unsubstituted or substituted phenylthio, unsubstituted or substituted phenylsulfinyl, unsubstituted or substituted phenylsulfonyl and unsubstituted and substituted or unsubstited cycloalkyl;
R₂, R₃, R₄, R₅, R₆, R₇, R₈, R₉ are independently selected from the group consisting of hydrogen, hydroxy, protected hydroxy, unsubstituted or substituted alkyl, unsubstituted or substituted alkenyl, unsubstituted or substituted alkynyl, unsubstituted or substituted cycloalkyl, unsubstituted or substituted cycloalkenyl, unsubstituted or substituted aryl, unsubstituted or substituted heteroaryl, unsubstituted or substituted heteroalicyclyl, unsubstituted or substituted heteroarylalkyl, unsubstituted or substituted heteroalicyclylalkyl, halogenated ether, nitro, amino, halogen, perhaloalkyl, —NX₁ₐX_{1b}, —CN, —C(=Z)X₁ₐ, —C(=Z)OX₁ₐ, —C(=Z)NX₁ₐX_{1b}, —N(X₁ₐ)—C(=Z)X_{1b}, —N(X₁ₐ)—C(=Z)NX_{1b}X_{1c}, —OC(=Z)X₁ₐ, —S(═O)X₁ₐ, —S(═O)₂X₁ₐ, and —SX₁ₐ, and,
R₁₀ is selected from the group consisting or oxygen, —NH or —NX_{1d} wherein:
Z is oxygen or sulfur; and,
X₁ₐ, X_{1b}, X_{1c} and X_{1d} are independently selected from the group consisting of hydrogen, substituted or unsubstituted aryl, substituted or unsubstituted heteroaryl, unsubstituted or substituted heteroalicyclyl, unsubstituted or substituted alkyl, unsubstituted or substituted alkenyl, unsubstituted or substituted alkynyl, unsubstituted or substituted cycloalkyl and unsubstituted or substituted cycloalkenyl.

2. The compound of claim 1, wherein:
R₁ is selected from the group consisting of hydrogen, unsubstituted or substituted C₁ to C₆ alkyl, unsubstituted or substituted phenyl, unsubstituted or substituted phenylalkyl, unsubstituted or substituted heteraryl, unsubstituted or substituted heteroalicyclyl, unsubstituted or substituted alkoxycarbonyl, unsubstituted or substituted C₁ to C₁₂ alkylaminocarbonyl, unsubstituted or substituted phenylaminocarbonyl, unsubstituted or substituted C₁ to C₁₀ alkylthio, unsubstituted or substituted phenylthio, and unsubstituted or substituted C₅ to C₇ cycloalkenyl.

3. The compound of claim 1 wherein:
R₂, R₃, R₄ and R₅ are independently selected from the group consisting of hydrogen, halogen, hydroxy, protected hydroxy, cyano, unsubstituted or substituted C₁ to C₁₂ alkyl, unsubstituted or substituted-C₁ to C₁₂ alkoxy, carboxy, protected carboxy, unsubstituted or substituted C₁ to C₁₀ alkylthio, —C(=Z)NX₁ₐX_{1b} and —C(=Z)X₁ₐ, wherein:
Z is oxygen or sulfur; and,
X₁ₐ and X_{1b} together with the nitrogen atom to which they are covalently bonded form an unsubstituted or substituted heteroaryl or an unsubstituted or substituted heteroalicyclyl; or,
X₁ₐ and X_{1b} are independently selected from the group consisting of hydrogen, unsubstituted or substituted C₁ to C₁₂ alkyl, unsubstituted or substituted phenyl, unsubstituted or substituted phenylalkyl, unsubstituted or substituted heteroaryl(C₁ to C₁₂)alkyl, unsubstituted or substituted heteroaryl, unsubstituted or substituted heteroalicyclyl(C₁ to C₁₂)alkyl and unsubstituted or substituted heteroalicyclyl;
R₆, R₇, R₈ and R₉ are independently selected from the group consisting of hydrogen, halogen, unsubstituted or substituted heteroaryl, unsubstituted or substituted heteroalicycly, —NX₁ₐX_{1b} and —SX₁ₐ, wherein X₁ₐ and X_{1b} are independently selected from the group consisting of hydrogen, unsubstituted or substituted C₁ to C₁₂ alkyl, unsubstituted or substituted C₂ to C₁₂ alkenyl, unsubstituted or substituted phenylalkyl, unsubstituted or substituted heteroaryl, unsubstituted or substituted heteroalicyclyl; and
R₁₀ is selected from the group consisting of oxygen, —NH or —NX_{1d} where X_{1d} is selected from the group consisting of hydrogen, substituted or unsubstituted aryl, substituted or unsubstituted heteroaryl, unsubstituted or substituted C₁ to C₁₀ alkyl, unsubstituted or substituted C₂ to C₁₀ alkenyl, unsubstituted or substituted C₂ to C₁₀ alkynyl, unsubstituted or substituted C₃ to C₈ cycloalkyl and unsubstituted or substituted C₅ to C₈ cycloalkenyl.

4. The compound of claim 1, wherein:
R₂, R₃, and R₅ are each hydrogen; and,
R₄ is —C(=Z)NX₁ₐX_{1b} or —C(=Z)X₁ₐ, wherein:
Z is oxygen or sulfur; and,
X₁ₐ and X_{1b} taken together with the nitrogen to which they are covalently bonded form a heterocycle or substituted heterocycle; or,
X₁ₐ and X_{1b} are independently selected from the group consisting of hydrogen, unsubstituted or substituted C₁ to C₁₂ alkyl, unsubstituted or substituted phenyl, unsubstituted or substituted heteroaryl, unsubstituted or substituted heteroalicyclyl;
R₆, R₈ and R₉ are each hydrogen;
R₇ is halogen, —NX₁ₐX_{1b} or heteroalicyclyl optionally substituted with —NX₁ₐX_{1b} or —SX₁ₐ, wherein:
X₁ₐ and X_{1b} are independently selected from the group consisting of hydrogen, unsubstituted or substituted C₁ to C₁₂ alkyl, unsubstituted or substituted C₂ to C₁₂ alkenyl, unsubstituted and substituted heteroaryl; and,
R₁₀ is selected from the group consisting of oxygen, —NH or NX_{1d} where X_{1d} is selected from the group consisting of hydrogen, substituted or unsubstituted aryl, substituted or unsubstituted heteroaryl, unsubstituted or substituted C₁ to C₁₀ alkyl, unsubstituted or substituted C₂ to C₁₀ alkenyl, unsubstituted or substituted C₂ to C₁₀ alkynyl, unsubstituted or substituted C₃ to C₁₀ cycloalkyl and unsubstituted or substituted C₅ to C₁₀ cycloalkenyl.

5. The compound of claim 1, wherein:
R₁ is selected from the group consisting of hydrogen, methyl, 2-propyl, 2-butyl, aminocarbonylethyl, 2-methylmercaptoethyl, phenyl, benzyl, cyclohexylmethyl, 4-methoxybenzyl, 4-chlorobenzyl, 3-indolylmethyl, 4-(trifluoroacetyl)aminobutyl;
R₂, R₃, R₅, R₆, R₈ and R₉ are each hydrogen; and,
R₄ is —C(=Z)NX₁ₐX_{1b} wherein:
Z is oxygen or sulfur; and,
X₁ₐ and X_{1b} together with the nitrogen to which they are covalently bonded form a group selected from the group consisting of 1-pyrrolidino, 4-methyl-1-homopiperazino, 4-(4-fluorophenyl)-1-piperazino, 4-(2-hydroxyethoxyethyl)-1-piperazino, 4-(2-pyridyl)-1-piperazino, 4-hydroxy-1-piperidino, 4-amino-2,2,6,6-tetramethyl-1-piperidino, 3-ethoxycarbonyl-1-piperidino, 4-(4-methoxyphenyl)-3-methyl-1-piperazino, 4-aminocarbonyl-1-piperidino, heptamethyleneimino, 4-(2-furoyl)-1-piperazino, 4-(3-trifluoromethylphenyl)-1-piperazino, 3-acetamido-1-pyrrolidino, 4-ethoxycarbonyl-1-piperazino, 4-ethoxycarbonyl-1-piperidino and 4-thiomorpholino, or
X₁ₐ is hydrogen and X_{1b} is selected from the group consisting of hydrogen, (1-ethyl-2-pyrrolidinyl)methyl, 2-thiazolyl, 5-methoxycarbonylpentyl, 2-ethoxy-carbonylethyl, 3-(methylthio)phenyl, N-methyl-(1-methyl-4-piperidino), 2-(pyridin-2-yl)ethyl, 2-hydroxyethyl, 3-(trifluoromethyl)benzyl, N,N-dimethylaminoethyl, 3-(2-oxo-1-pyrrolidino)propyl, 1-ethoxycarbonyl-4-piperidino, pyridin-2-ylmethyl, bis(2-methoxyethyl), 2-acetylaminoethyl, 3-(methylthio)propyl, 2-(1-morpholino)ethyl, 5-indazolyl, cyclopropyl, N-ethyl-(pyridin-4-ylmethyl), cyclopentyl, cycloheptyl, pyridin-3-ylmethyl, 4-(trifluoromethyl)benzyl, 2-(thien-2-yl)ethyl, 3-(N-pyrrolidino)propyl or 3-(1-imidazolyl)propyl; and
R₇ is selected from the group consisting of cyclopropylamino, 2-(1-morpholino)ethylamino, piperazino, 2-methyl-4-(3-methylphenyl )-1-piperazino, 4-aminocarbonylpiperidino, 2-(pyridin-2-yl )ethylamino, 2-(N,N-dimethylamino)ethylamino, 3-(aminomethyl)benzylamino, (5-phenyl-1H-1,2,4-triazol-3-yl)thio, 3-(4-morpholino)propylamino, tetrahydrofurfurylamino, 4-(2,5-dimethylphenyl)-1-piperazino, hexamethyleneimino, N-methyl-2-(pyridin-2-yl)ethylamino, 2-(dimethylamino)ethylamino, 4-(aminomethyl)benzylamino, (3-carboxypyridin-6-yl)thio, 2-acetylaminoethylamino, 2-(ethoxycarbonyl)-ethylamino, 4-(2,3-dimethylphenyl)-1-piperazino, 4-(2-pyridyl)-1-piperazino, 3-(2-pipecolino)propylamino, 2-aminoethylamino, cyclohexylamino, imidazol-2-ylthio, 4-ethoxycarbonyl-1-piperazino, 3-methylthiopropylamino, 4-(4-fluorophenyl)piperazino, 1-benzyl-3-pyrrolidinoamino, N-methyl-4-piperidylamino, 3-aminopropylamino, N-benzylmethylamino, (3,5-dimethyl-2,6-pyrimidin-2-yl)thio, 4-acetyl-1-piperazino, 2,3-dimethoxybenzylamino, 4-(3,4-dichlorophenyl )-1-piperazino, 3-ethoxycarbonyl-1-piperidino, pyridin-3-ylmethylamino, N-methyl-2-(diethylamino)ethylamino, N-methylphenethylamino, (5-methyl-1,3,4-thiadiazol-2-yl)thio, 8-amino-3,6-dioxaoctyamino, 3-acetamido-1-pyrrolidino, 4-benzyl-1-piperazino, 4-ethoxycarbonyl-1-piperazino, 2-piperadinoethylamino, 3-dimethylaminopropylamino, cycloheptylamino, (1H-1,2,4-triazol-3-yl)thio, 4-ethoxycarbonylmethyl-1-piperazino, 4-(diethylamino)-2-butenylamino, 4-(4-nitrophenyl)-1-piperazino, 1-ethoxycarbonyl-4-piperidylamino, 1-benzyl-4-piperidylamino, N-methyl-3-(dimethylamino)propylamino, 4-(trifluoromethyl)benzylamino, (4-methyl-1,2,4-triazol-3-yl)thio, 2-ethoxyethylamino, tyramino, 4-(3-trifluoromethylphenyl )-1-piperazino, 1,3,3-trimethyl-6-aza-6-bicyclo(3,2,1)-octyl, 3,3′-bis(dimethylamino)dipropylamino, butylamino, 3-(trifluoromethyl)benzylamino, pyridin-2-ylthio, 4-(2-furoyl)-1-piperazino, cyclooctylamino, 4-(4-acetylphenyl)-1-piperazino, 4-(4-methylphenyl)-3-methyl-1-piperazino, 2-fluorophenethylamino, 3-fluorophenethylamino, 4-fluorobenzylamino, fluoro, morpholino, thiomorpholino, 4-(5-chloro-2-methylphenyl )-1-piperazino, (1-ethyl-2-pyrrolidino)methylamino, 2,2,6,6-tetramethyl-4-piperidylamino, diethylamino and 3,3,5-trimethylcyclo-hexyamino.

6. The compound of claim 1, wherein:
R₁ is selected from the group consisting of hydrogen, methyl, 2-propyl, 2-butyl, aminocarbonylethyl, 2-methylmercaptoethyl, phenyl, benzyl, cyclohexylmethyl, 4-methoxybenzyl, 4-chlorobenzyl, 3-indolylmethyl, 4-(trifluoroacetyl)aminobutyl and 3-guanidinopropyl;
R₂, R₃, R₄ and R₅ are independently selected from the group consisting of hydrogen, methyl, carboxy, bromo, fluoro, chloro and trifluoromethyl;
R₆, R₈ and R₉ are each hydrogen;
R₇ is selected from the group consisting of cyclopropylamino, 2-(1-morpholino)ethylamino, piperazino, 2-methyl-4-(3-methylphenyl )-1-piperazino, 4-aminocarbonylpiperidino, 2-(pyridin-2-yl)ethylamino, 2-(N,N-dimethylamino)ethylamino, 3-(aminomethyl)-benzylamino, (5-phenyl-1H-1,2,4-triazol-3-yl)thio, 3-(4-morpholino)-propylamino, tetrahydrofurfurylamino, 4-(2,5-dimethylphenyl)-1-piperazino, hexamethyleneimino, N-methyl-2-(pyridin-2-yl)ethylamino, 2-(dimethylamino)ethylamino, 4-(aminomethyl)benzylamino, (3-carboxypyridin-6-yl)thio, 2-acetylaminoethylamino, 2-(ethoxycarbonyl)-ethylamino, 4-(2,3-dimethylphenyl)-1-piperazino, 4-(2-pyridyl)-1-piperazino, 3-(2-pipecolino)propylamino, 2-aminoethylamino, cyclohexylamino, imidazol-2-ylthio, 4-ethoxycarbonyl-1-piperazino, 3-methylthiopropylamino, 4-(4-fluorophenyl)piperazino, 1-benzyl-3-pyrrolidinoamino, N-methyl-4-piperidylamino, 3-aminopropylamino, N-benzylmethylamino, (3,5-dimethyl-2,6-pyrimidin-2-yl)thio, 4-acetyl-1-piperazino, 2,3-dimethoxybenzylamino, 4-(3,4-dichlorophenyl)-1-piperazino, 3-ethoxycarbonyl-1-piperidino, pyridin-3-ylmethylamino, N-methyl-2-(diethylamino)ethylamino, N-methylphenethyl-amino, (5-methyl-1,3,4-thiadiazol-2-yl)thio, 8-amino-3,6-dioxaoctyamino, 3-acetamido-1-pyrrolidino, 4-benzyl-1-piperazino, 4-ethoxycarbonyl-1-piperazino, 2-piperadino-ethylamino, 3-dimethylaminopropylamino, cycloheptylamino, (1H-1,2,4-triazol-3-yl)thio, 4-ethoxycarbonylmethyl-1-piperazino, 4-(diethylamino)-2-butenylamino, 4-(4-nitrophenyl)-1-piperazino, 1-ethoxycarbonyl4-piperidylamino, 1-benzyl-4-piperidylamino, N-methyl-3-(dimethylamino)propylamino, 4-(trifluoromethyl)benzylamino, (4-methyl-1,2,4-triazol-3-yl)thio, 2-ethoxyethylamino, tyramino, 4-(3-trifluoromethylphenyl)-1-piperazino, 1,3,3-trimethyl-6-aza-6-bicyclo(3,2,1)-octyl, 3,3′-bis(dimethyl-amino)dipropylamino, butylamino, 3-(trifluoromethyl)benzylamino, pyridin-2-ylthio, 4-(2-furoyl)-1-piperazino, cyclooctylamino, 4-(4-acetylphenyl)-1-piperazino, 4-(4-methylphenyl)-3-methyl-1-piperazino, 2-fluorophenethyl-amino, 3-fluorophenethylamino, 4-fluorobenzylamino, fluoro, morpholino, thiomorpholino, 4-(5-chloro-2-methylphenyl 1-piperazino, (1-ethyl-2-pyrrolidino)methylamino, 2,2,6,6-tetramethyl-4-piperidylamino, diethylamino and 3,3,5-trimethylcyclo-hexyamino; and,
R₁₀ is selected from the group consisting or oxygen, —NH and —NX_{1d} wherein:
X_{1d} is selected from the group consisting of hydrogen, unsubstituted or substituted aryl, unsubstituted or substituted heteroaryl, unsubstituted or substituted C₁ to C₁₀ alkyl, unsubstituted or substituted C₂ to C₁₀ alkenyl, unsubstituted or substituted C₂ to C₁₀ alkynyl, C₃ to C₁₀ cycloalkyl and C₅ to C₁₀ cycloalkenyl.

7. A compound having a chemical structure selected from the group consisting of:

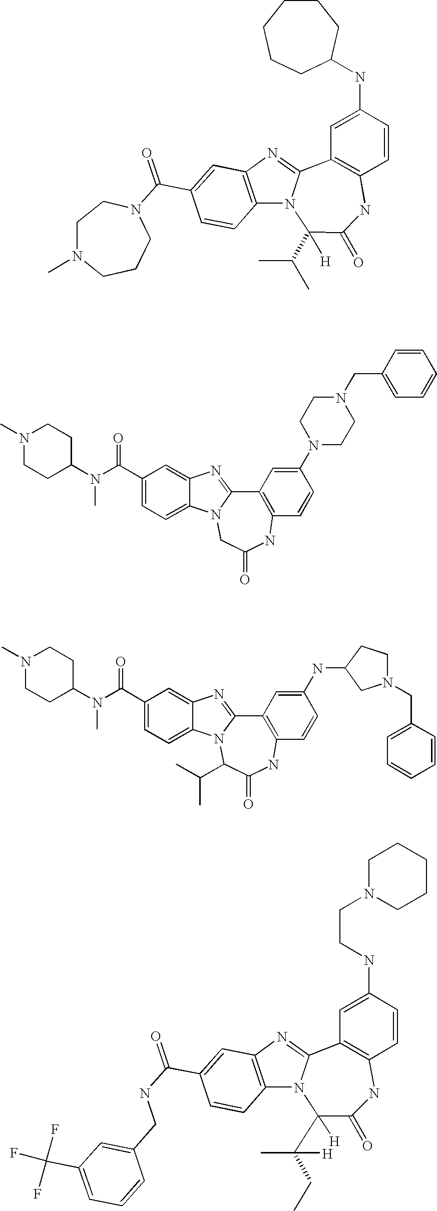

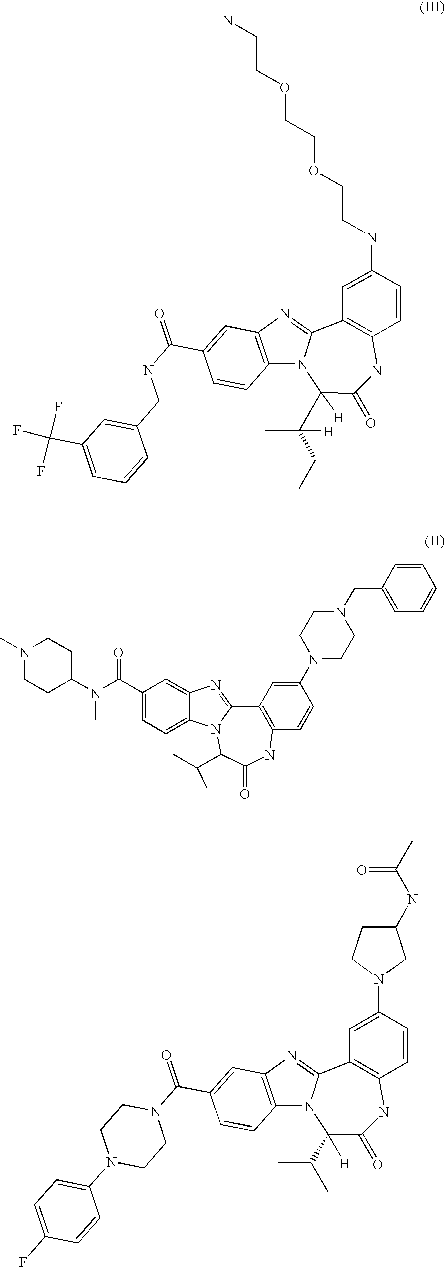

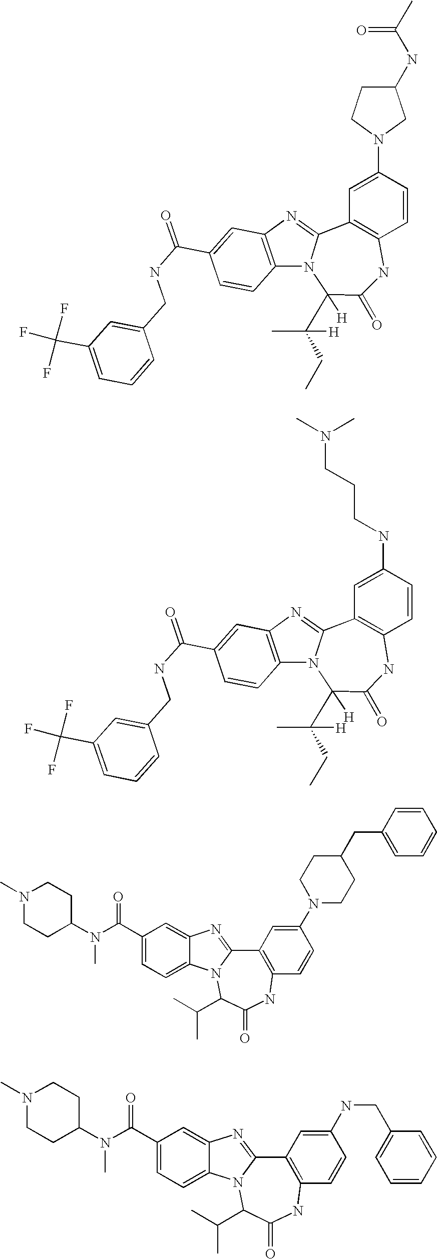

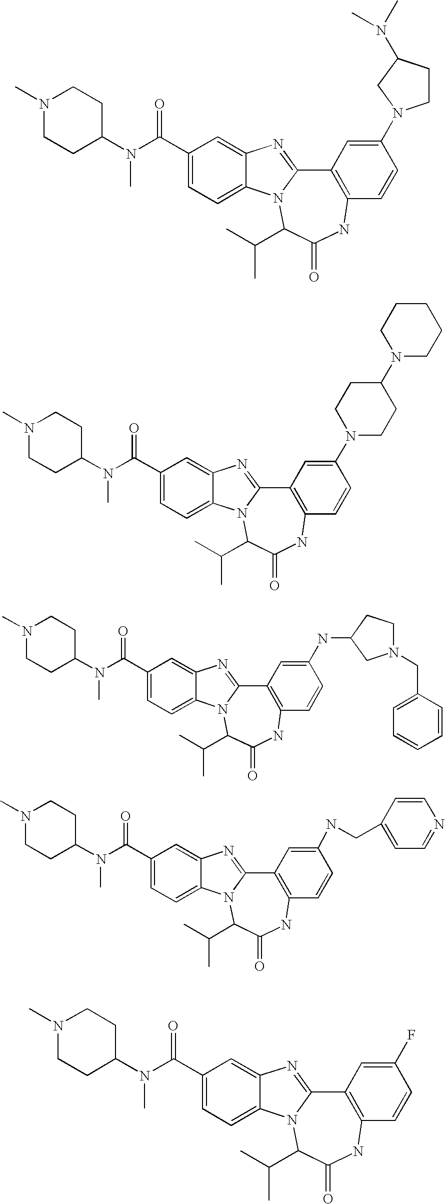

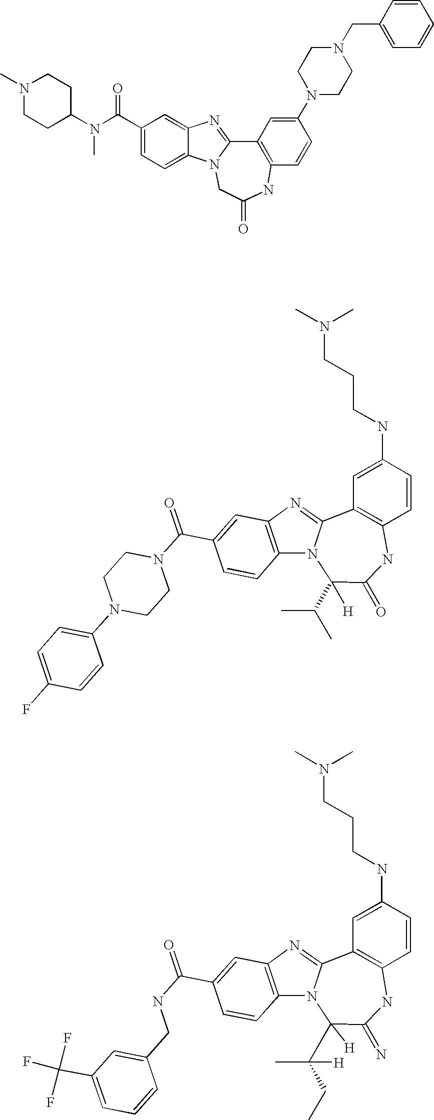

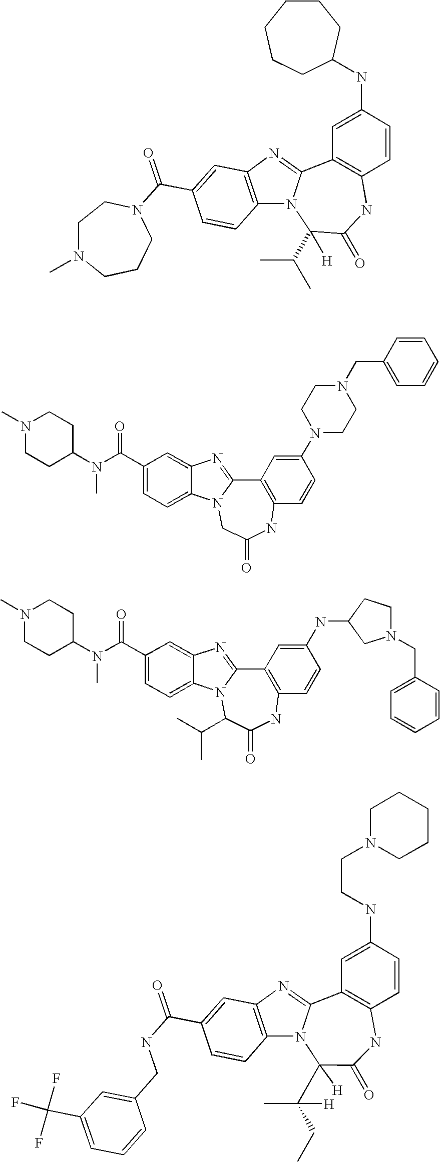

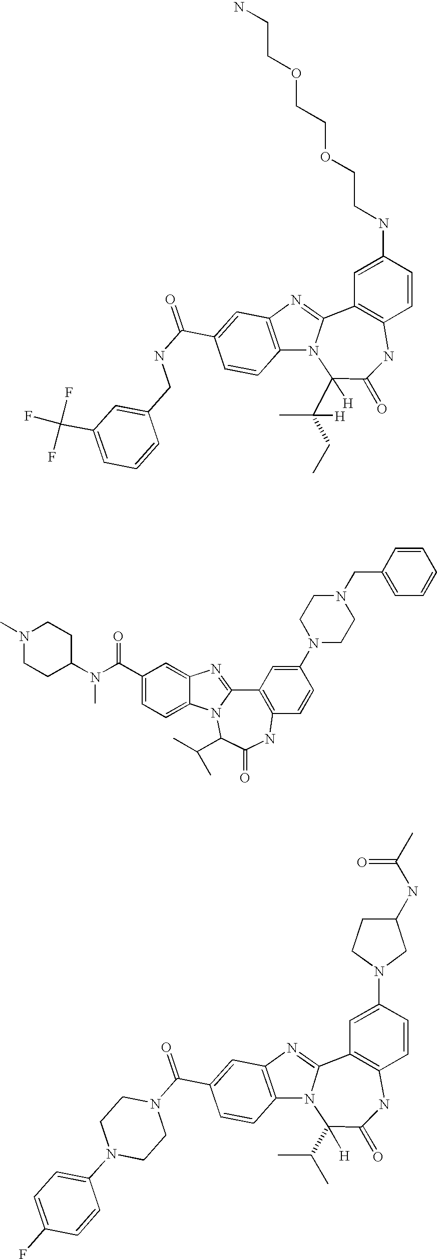

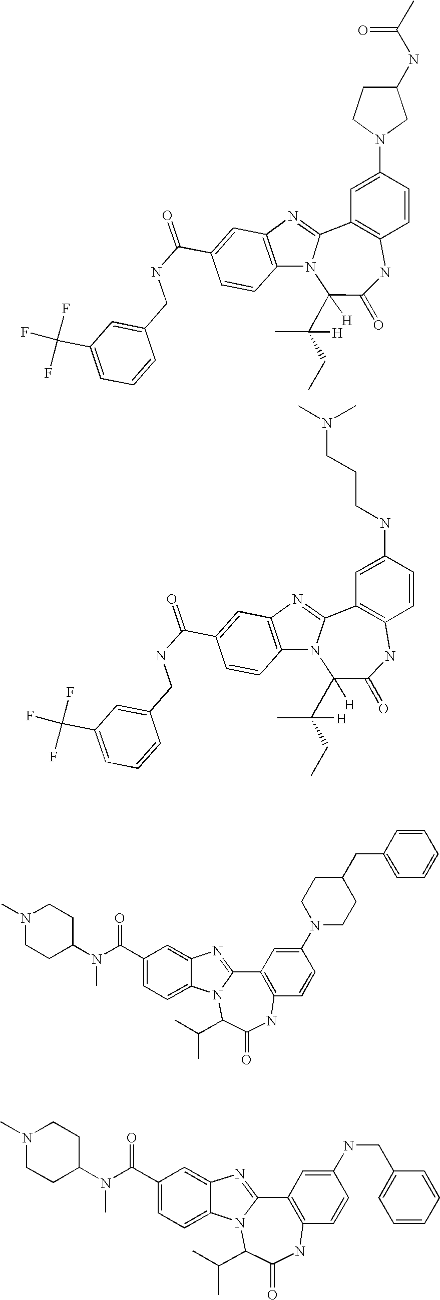

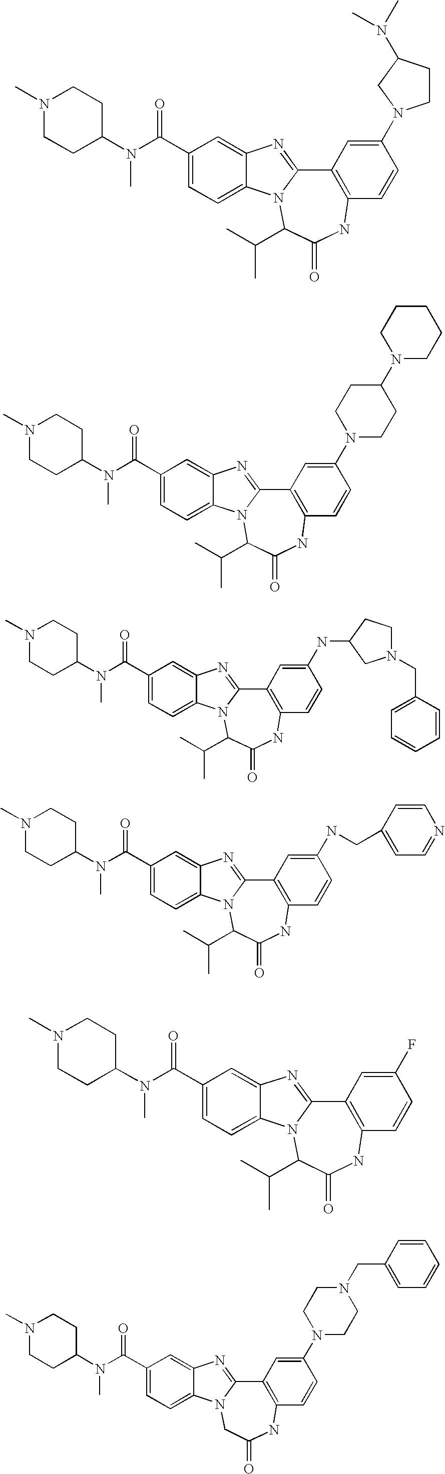

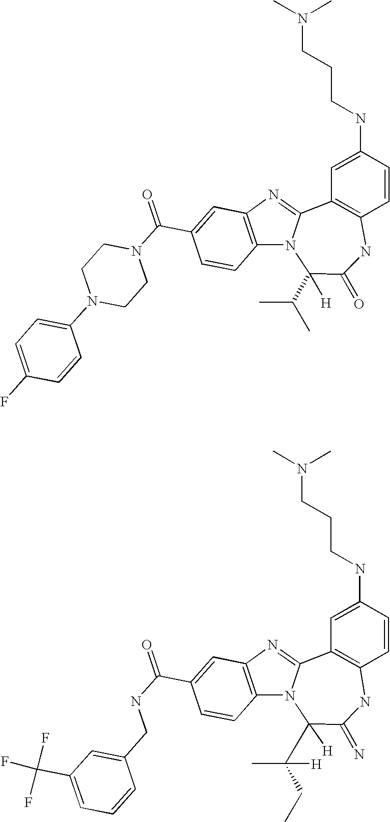

8. A method of treating or preventing pain, comprising administering to a patient in need thereof an effective amount of at least one compound of this invention, or a salt or prodrug thereof.

9. The method of claim 8, wherein the pain is associated with diabetes, viral infection, irritable bowel syndrome, amputation, cancer, acute or chronic inflammation, arthritis, physical trauma or the side effects of therapeutic drugs.

10. A method of screening for a compound that modulates the activity of a human MrgX1, a simian MrgX1, a human MrgX2 or a simian MrgX2 receptor comprising:
contacting a test compound with a recombinant cell comprising a recombinant nucleic acid that expresses the human MrgX1, the simian MrgX1, the human MrgX2 or the simian MrgX2 receptor, provided that the cell does not contain an endogenous nucleic acid that expresses the functional receptor; and,
detecting changes in the activity of the receptor.

11. The method of claim 10, wherein the effect of the test compound on the cell or plurality of cells that express(es) the human MrgX1, the simian MrgX1, the human MrgX2 or the simian MrgX2 receptor is compared to its affect on the non-recombinant cell or plurality of cells, wherein:
if the compound has no effect on the non-recombinant cell(s) but exhibits an effect on the recombinant cell(s), the compound is a specific agonist, inverse agonist or antagonist of the receptor.

12. The method of claim 10, wherein the recombinant nucleic acid is selected from the group consisting of:
a nucleic acid of SEQ ID NO:1;
a nucleic acid encoding the amino acid SEQ ID NO:2;
a nucleic acid of SEQ ID NO:3;
a nucleic acid encoding the amino acid SEQ ID NO:4;
a nucleic acid of SEQ ID NO:5;
a nucleic acid encoding the amino acid SEQ ID NO:6;
a nucleic acid of SEQ ID NO:7;
a nucleic acid encoding the amino acid SEQ ID NO:8;
a nucleic acid of SEQ ID NO:9;
a nucleic acid encoding the amino acid SEQ ID NO:10;
a nucleic acid of SEQ ID NO:11;
a nucleic acid encoding the amino acid of SEQ ID NO:12;
a nucleic acid of SEQ ID NO: 13;
a nucleic acid encoding the amino acid of SEQ ID NO:14;
a nucleic acid of SEQ ID NO 15; and,
a nucleic acid encoding the amino acid of SEQ ID NO:16.

13. The method of claim 10, further comprising a nucleic acid that is at least 85% homologous with any one of SEQ ID NO:1, SEQ ID NO:3, SEQ ID NO:5, SEQ ID NO:7, SEQ ID NO:9, SEQ ID NO:11, SEQ ID NO. 13 and SEQ ID NO:15, and that encodes an amino acid sequence that exhibits substantially the same activity as the human MrgX1, the simian MrgX1, the human, MrgX2 or the simian MrgX2 receptor encoded by the aforementioned nucleic acid sequences.
